(19) United States
(12) Patent Application Publication
(10) Pub. No.: US 2012/0053349 A1
(43) Pub. Date: Mar. 1, 2012

(54) OXAMIDE DERIVATIVE

(75) Inventors: Toshiharu Yoshino, Tokyo (JP); Yumi Nakamoto, Tokyo (JP); Tomoyuki Nagai, Kanagawa (JP); Seishiro Uchida, Tokyo (JP)

(73) Assignee: DAIICHI SANKYO COMPANY, LIMITED, Tokyo (JP)

(21) Appl. No.: 13/273,360

(22) Filed: Oct. 14, 2011

Related U.S. Application Data
(63) Continuation-in-part of application No. PCT/JP2010/057990, filed on May 12, 2010.

(30) Foreign Application Priority Data
May 15, 2009 (JP) 2009-119274

(51) Int. Cl. C07D 471/04

(52) U.S. Cl. 546/114

(57) ABSTRACT

The present invention provides an isomer of N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by formula (II) or a salt thereof, or a solvate thereof. Such an isomer or a salt thereof, or a solvate thereof is useful as a standard in a test for inspecting impurities in a pharmaceutical composition containing a compound represented by formula (II) or a salt thereof, or a hydrate thereof. The present invention also relates to a substantially pure compound that is useful as a preventive and/or therapeutic drug for thrombotic diseases, and a pharmaceutical composition containing the substantially pure compound.

This application is a continuation-in-part of International Application No. PCT/JP2010/057990, filed on May 12, 2010, entitled “OXAMIDE DERIVATIVE”, which claims the benefit of Japanese Patent Application Number JP 2009-119274, filed on May 15, 2009, all of which are hereby incorporated by reference in their entirety.

### FIELD OF THE INVENTION

The present invention relates to an isomer of a compound that exhibits an inhibitory effect on activated blood coagulation factor X, and is useful as a preventive and/or therapeutic drug for thrombotic diseases. The present invention also relates to a substantially pure compound that is useful as a preventive and/or therapeutic drug for thrombotic diseases, and a pharmaceutical composition containing the substantially pure compound. Specifically, the present invention relates to a substantially pure compound II or salt thereof, or solvate thereof which substantially free from isomer of compound II or salt thereof, or solvate thereof. The present invention also relates to a substantially pure pharmaceutical composition containing compound II or salt thereof, or solvate thereof, which substantially free from isomer of compound II or salt thereof, or solvate thereof.

### BACKGROUND OF THE INVENTION

N¹-(5-Chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (II) (in the present specification, also referred to as compound II):

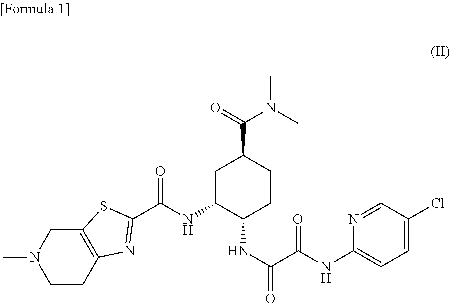

or a salt thereof, or a solvate thereof, for example, p-toluenesulfonic acid monohydrate of compound II represented by the following formula (I) (in the present specification, also referred to as compound I):

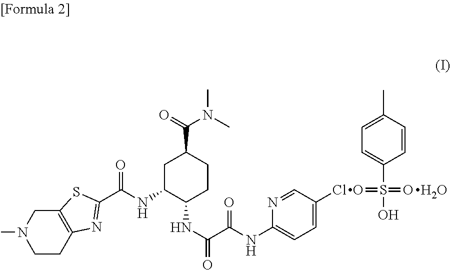

is known as a compound that exhibits an inhibitory effect on activated blood coagulation factor X (also referred to as activated factor X or FXa), and is useful as a preventive and/or therapeutic drug for thrombotic diseases.

A stereoisomer in which three substituents on a cyclohexyl group have absolute configuration of 1R,2R,4S is known as a stereoisomer of compound II (Example 269 of Patent Literature 1).

### CITATION LIST

### Patent Literature

(Patent Literature 1) International Publication No. WO 03/000657
(Patent Literature 2) International Publication No. WO 03/000680
(Patent Literature 3) International Publication No. WO 03/016302
(Patent Literature 4) International Publication No. WO 05/047296
(Patent Literature 5) International Publication No. WO 07/032,498

### SUMMARY OF INVENTION

### Technical Problem

An object of the present invention is to provide an isomer of compound II or a salt thereof, or a solvate thereof, which can be used as a standard in a test for inspecting impurities in pharmaceutical drugs containing compound II or a salt thereof, or a solvate thereof, to provide substantially pure compound II or a salt thereof, or a solvate thereof, which is substantially free from an isomer or a salt thereof, or a solvate thereof as impurities, and to provide substantially pure pharmaceutical composition containing compound II or a salt thereof, or a hydrate thereof, which is substantially free from an isomer or a salt thereof, or a solvate thereof as impurities.

### Solution to Problem

The present invention relates to a stereoisomer of compound II, specifically:

[1] N¹-(5-chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (III) (in the present specification, also referred to as compound III):

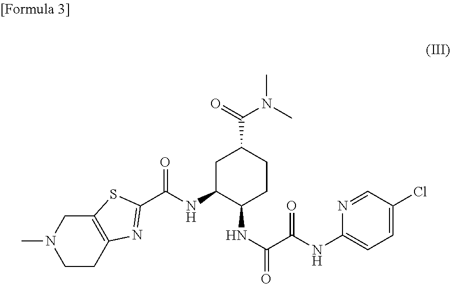

or a salt thereof, or a solvate thereof;
[2] N¹-(5-chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IV) (in the present specification, also referred to as compound IV):

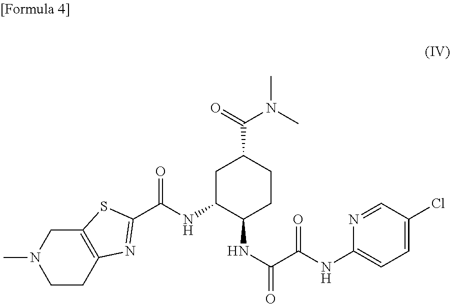

or a salt thereof, or a solvate thereof;
[3] N¹-(5-chloropyridin-2-yl)-N²-((1R,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (V) (in the present specification, also referred to as compound V):

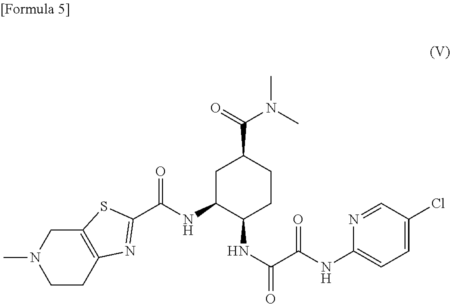

or a salt thereof, or a solvate thereof;
[4] N¹-(5-chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VI) (in the present specification, also referred to as compound VI):

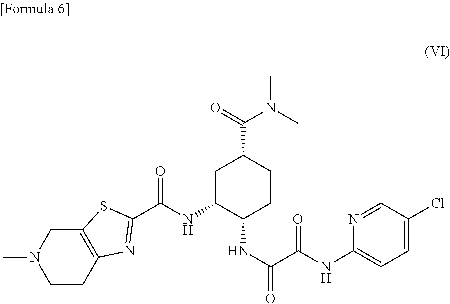

or a salt thereof, or a solvate thereof;
[5] N¹-(5-chloropyridin-2-yl)-N²-((1S,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VII) (in the present specification, also referred to as compound VII):

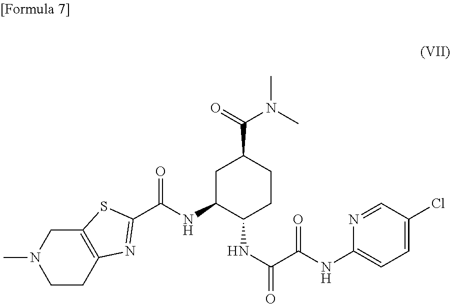

or a salt thereof, or a solvate thereof;
[6] N¹-(5-chloropyridin-2-yl)-N²-((1S,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VIII) (in the present specification, also referred to as compound VIII):

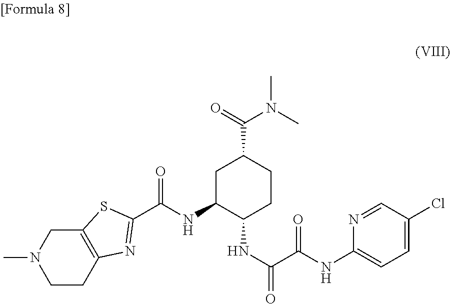

or a salt thereof, or a solvate thereof;
[7] a method for inspecting impurities in a pharmaceutical composition containing compound II or a salt thereof, or a hydrate thereof, wherein the method comprises using, as a standard, at least one or more compound(s), salt(s), or solvate(s) selected from the group consisting of N¹-(5-chloropyridin-2-yl)-N²-((1R,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IX):

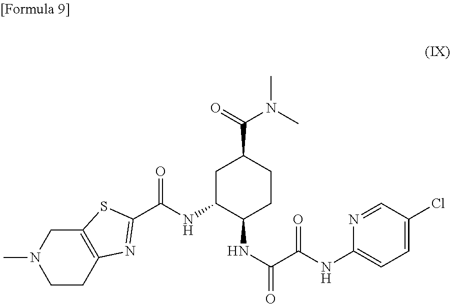

(in the present specification, also referred to as compound IX) or a salt thereof, or a solvate thereof, and a compound or a salt thereof, or a solvate thereof according to any of [1] to [6];
[8] substantially pure compound II or a salt thereof, or a solvate thereof, wherein the compound II or salt, or solvate thereof is substantially free from isomer of compound II or salt thereof, or solvate thereof;
[9] the compound or a salt thereof, or a solvate thereof according to [8], wherein the content of the isomer or salt thereof, or solvate thereof is 3% or less;
[10] a substantially pure pharmaceutical composition containing compound II or a salt thereof, or a solvate thereof, wherein the composition is substantially free from isomer of compound II or salt thereof, or solvate thereof; and/or
[11] the pharmaceutical composition according to [10], wherein the content of the isomer or salt thereof, or solvate thereof is 3% or less.

### Advantageous Effects of Invention

There is provided an isomer of compound II that is useful as a pharmaceutical compound or a salt thereof, or a solvate thereof. Such an isomer or a salt thereof, or a solvate thereof can be used as a standard in the inspection of impurities in a pharmaceutical composition containing compound II or a salt thereof, or a solvate thereof. There is also provided substantially pure compound II or a salt thereof, or a solvate thereof substantially free from isomer of compound II or a salt thereof, or a solvate thereof as impurities.

### DETAILED DESCRIPTION

Hereinafter, the present invention will be described in detail.

N¹-(5-Chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (II):

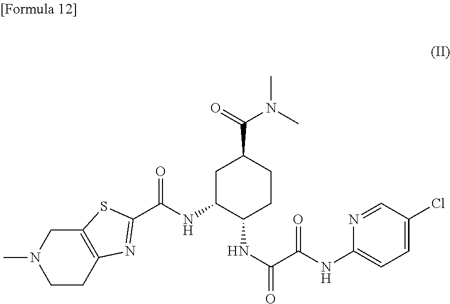

is the free form of compound I and is called edoxaban (N-(5-chloropyridin-2-yl)-N′-[(1S,2R,4S)-4-(N,N-dimethylcarbamoyl)-2-(5-methyl-4,5,6,7-tetrahydro[1,3]thiazolo[5,4-c]pyridine-2-carboxamido)cyclohexyl]oxamide) as International Nonproprietary Name (INN).

In the present specification, the term “solvate” means a solvate which may be formed by leaving the compound of the present invention in a solvent or recrystallizing the compound of the present invention. In the present specification, the solvate encompasses hydrates.

The compound of the present invention may contain normatural proportions of atomic isotopes for one or more of atoms constituting such a compound. Examples of the atomic isotopes include heavy hydrogen (²H), tritium (³H), and carbon-14 (¹⁴C). Moreover, the compound may be radiolabeled with, for example, a radioisotope such as tritium (³H) or carbon-14 (¹⁴C). The radiolabeled compound is useful as a therapeutic or preventive agent, a research reagent, for example, an assay reagent, and a diagnostic agent, for example, an in vivo diagnostic imaging agent. All isotopic variants of the compound of the present invention are incorporated in the scope of the present invention, regardless of whether they are radioactive or not.

Compound II or a salt thereof, or a solvate thereof (e.g., compound I), and compound IX can be synthesized according to a method described in, for example, Patent Literatures 1 to 3, though the synthesis method is not limited thereto.

Compounds III to VIII of the present invention can be synthesized according to a method described in Examples, though the synthesis method is not limited thereto. Compounds III to VIII of the present invention can be synthesized using an organic chemical method usually performed by those skilled in the art with reference to the description of Examples.

In the present specification, the term “isomer” refers to an isomer of compound II and specifically refers to compounds III to IX, unless otherwise specified.

A solvent used in reaction in each step of the synthesis of compounds III to VIII of the present invention is not particularly limited and can be any solvent that does not inhibit the reaction and dissolves starting materials to some extent. Examples of the solvent include: hydrocarbons such as pentane, hexane, octane, petroleum ether, ligroin, and cyclohexane; amides such as formamide, N,N-dimethylformamide, and N,N-dimethylacetamide; ethers such as diethyl ether, diisopropyl ether, tetrahydrofuran, dioxane, dimethoxyethane, and diethylene glycol dimethyl ether; alcohols such as methanol, ethanol, n-propanol, n-butanol, 2-butanol, and 2-methyl-1-propanol; sulfoxides such as dimethyl sulfoxide; sulfones such as sulfolane; nitriles such as acetonitrile, propionitrile, butyronitrile, and isobutyronitrile; esters such as ethyl formate, ethyl acetate, and propyl acetate; ketones such as acetone and methyl ethyl ketone; nitro compounds such as nitroethane and nitrobenzene; halogenated hydrocarbons such as dichloromethane, 1,2-dichloroethane, chlorobenzene, dichlorobenzene, chloroform, and carbon tetrachloride; aromatic hydrocarbons such as benzene, toluene, and xylene; carboxylic acids such as acetic acid, formic acid, propionic acid, butyric acid, and trifluoroacetic acid; amines such as N-methylmorpholine, triethylamine, tripropylamine, tributylamine, diisopropylethylamine, and piperidine; water; and mixed solvents thereof.

A base used in reaction in each step of the synthesis of compounds III to VIII of the present invention is not particularly limited, and examples thereof include: inorganic bases such as alkali metal carbonates (e.g., sodium carbonate and potassium carbonate), alkali metal bicarbonates (e.g., sodium bicarbonate, potassium bicarbonate, and lithium bicarbonate), alkali metal acetates (e.g., sodium acetate and potassium acetate), alkali metal hydrides (e.g., lithium hydride, sodium hydride, and potassium hydride), alkali metal hydroxides (e.g., sodium hydroxide, potassium hydroxide, barium hydroxide, and lithium hydroxide), and alkali metal fluorides (e.g., sodium fluoride and potassium fluoride); alkali metal alkoxides such as sodium methoxide, sodium ethoxide, and sodium-t-butoxide; organic bases such as 1,5-diazabicyclo[4.3.0]non-5-ene (DBN), and 1,8-diazabicyclo[5.4.0]undec-7-ene (DBU); organic metal bases such as n-butyllithium, lithium diisopropylamide, and lithium bis(trimethylsilyl)amide; and amino acids such as proline.

A condensing agent used in reaction in each step of the synthesis of compounds III to VIII of the present invention is not particularly limited, and examples thereof include 1,3-dicyclohexylcarbodiimide, isobutyl chloroformate, pivalic acid chloride, isovaleric acid chloride, 1-ethyl-3-(3-dimethylaminopropyl)carbodiimide, 1-cyclohexyl-3-morpholinoethyl carbodiimide, 1-cyclohexyl-3-(4-diethylaminocyclohexyl)carboximide, N,N′-carbonyldiimidazole, 2-chloro-1,3-dimethylimidazolinium chloride, and isobutyl chloroformate.

The reaction temperature of reaction in each step of the synthesis of compounds III to VIII of the present invention differs depending on solvents, starting materials, reagents, etc., and the reaction time thereof differs depending on solvents, starting materials, reagents, the reaction temperature, etc.

After the completion of reaction in each step of the synthesis of compounds III to VIII of the present invention, each compound of interest is collected from the reaction mixture according to a routine method. For example, the reaction mixture is appropriately neutralized, or insoluble matter, if any, is removed by filtration, and an organic layer containing the compound of interest is then separated by the addition of water and water-immiscible organic solvent such as ethyl acetate, washed with water or the like, and then dried over anhydrous magnesium sulfate, anhydrous sodium sulfate, anhydrous sodium bicarbonate, or the like. After filtration, the solvent is distilled off to obtain the compound of interest. The obtained compound of interest can be separated and purified, if necessary, by a routine method, for example, by appropriately combining usual methods commonly used in separation and purification of organic compounds such as recrystallization and reprecipitation, applying chromatography, and eluting the compound with an appropriate eluent. The compound of interest that is insoluble in a solvent can be purified by washing a crude product of the obtained solid with a solvent. Moreover, the compound of interest in each step may be used directly in the next reaction without being purified.

For using the compound of interest as a pharmaceutical drug, the contamination of a bulk pharmaceutical and a preparation containing the same with impurities is not preferable, and the profile of impurities in pharmaceutical drugs is strictly controlled by the setting of specification tests. When at least one or more compound(s), salt(s), or solvate(s) selected from compounds III to IX of the present invention or salts thereof, or solvates thereof is used as a standard in a test for inspecting impurities in a pharmaceutical composition containing compound II or a salt thereof, or a solvate thereof, the method is not particularly limited as long as it is a test for inspecting impurities usually used by those skilled in the art. Examples thereof include methods using HPLC. The test for inspecting impurities in a pharmaceutical composition containing compound II or a salt thereof, or a solvate thereof can be carried out by first examining the physicochemical properties of at least one or more compound(s), salt(s), or solvate(s) selected from compounds III to IX or salts thereof, or solvates thereof (e.g., examining HPLC peaks characteristic of the compound(s), salt(s), or solvate(s) using an HPLC method) and next confirming the presence or absence of these physicochemical properties for the pharmaceutical composition containing compound II, a pharmaceutically acceptable salt thereof, or a solvate thereof.

Compound I produced according to a method described in WO 2005/047296 or WO 2007/032498 or the like contained with regard to enantiomer compound III 0.03% or less, and with regard to compound IV to IX, only 0.01% or less in terms of the total amount of compounds IV and VII, only 0.01% or less in terms of the total amount of compounds V and VI, and only 0.01% or less in terms of the total amount of compounds VIII and IX.

The present invention relates to substantially pure compound II or a salt thereof, or a solvate thereof with a low content of an isomer or a salt thereof, or a solvate thereof as impurities. The present invention also relates to substantially pure compound II or a salt thereof, or a solvate thereof having an isomer or a salt thereof, or a solvate thereof content of 3% or less, 2% or less, 1% or less, 0.9% or less, 0.8% or less, 0.7% or less, 0.6% or less, 0.5% or less, 0.4% or less, 0.3% or less, 0.2% or less, 0.1% or less, 0.09% or less, 0.08% or less, 0.07% or less, 0.06% or less, 0.05% or less, or 0.04% or less.

The present invention relates to substantially pure compound I with a low content of an isomer as impurities. The present invention also relates to substantially pure compound I having an isomer content of 3% or less, 2% or less, 1% or less, 0.9% or less, 0.8% or less, 0.7% or less, 0.6% or less, 0.5% or less, 0.4% or less, 0.3% or less, 0.2% or less, 0.1% or less, 0.09% or less, 0.08% or less, 0.07% or less, 0.06% or less, 0.05% or less, or 0.04% or less.

Substantially pure compound I with a low content of an isomer may be produced according to a method described in WO 2005/047296 or WO 2007/032498 or the like or can also be obtained by synthesizing compound I according to a method described in WO 2005/047296 or WO 2007/032498 or the like or a method other than the method described in WO 2005/047296 or WO 2007/032498 or the like and then purifying the obtained compound by a purification method that would be apparent to those skilled in the art (e.g., column chromatography, recrystallization, or a combination thereof).

In the present specification, the content of impurities such as compounds III to IX is indicated in percentage (%) by area measured by liquid chromatography and can be determined by a method described in, for example, Example 7 described later.

The present invention also relates to a pharmaceutical composition containing such substantially pure compound II or a salt thereof, or a solvate thereof (e.g., substantially pure compound I as described above). Compound II or a salt thereof, or a solvate thereof (e.g., compound I) exhibits a high inhibitory effect on activated blood coagulation factor X (also referred to as FXa) and as such, is useful as an anticoagulant agent or a preventive and/or therapeutic agent for thrombus or embolism. Compound II or a salt thereof, or a solvate thereof (e.g., compound I) is useful as a pharmaceutical drug for mammals including humans, an activated blood coagulation factor X inhibitor, an anticoagulant agent, a preventive and/or therapeutic agent for thrombus and/or embolism, a preventive and/or therapeutic agent for thrombotic disease, and further a preventive (in the present specification, the prevention encompasses secondary prevention) and/or therapeutic agent for cerebral infarction, cerebral embolism, pulmonary infarction, pulmonary embolism, myocardial infarction, angina pectoris, thrombus and/or embolism accompanying non-valvular atrial fibrillation (NVAF), deep vein thrombosis, deep vein thrombosis after surgical operation, thrombosis after prosthetic valve/joint replacement, thromboembolism after total hip replacement (THR), thromboembolism after total knee replacement (TKR), thromboembolism after hip fracture surgery (HFS), thrombosis and/or reocclusion after revascularization, Buerger's disease, disseminated intravascular coagulation syndrome, systemic inflammatory response syndrome (SIRS), multiple organ dysfunction syndrome (MODS), thrombosis at the time of extracorporeal circulation, or blood coagulation at the time of blood collection, or a bulk pharmaceutical for these preventive and/or therapeutic agents.

The compound of the present invention or a pharmaceutically acceptable salt thereof, or a solvate thereof is also useful as an anticoagulant agent or a preventive and/or therapeutic agent for thrombus or embolism.

A pharmaceutical drug comprising the compound of the present invention or a pharmaceutically acceptable salt thereof, or a solvate thereof as an active ingredient is preferably provided in the form of a pharmaceutical composition comprising the compound of the present invention or a pharmaceutically acceptable salt thereof, or a solvate thereof and one or two or more pharmaceutically acceptable carriers. The dosage form of the pharmaceutical drug of the present invention is not particularly limited. It can be administered orally or parenterally and is preferably administered orally.

Examples of the pharmaceutically acceptable carrier used in the production of the pharmaceutical composition comprising the compound of the present invention or a pharmaceutically acceptable salt thereof, or a solvate thereof as an active ingredient can include, but are not limited to, excipients, disintegrants or disintegration aids, binders, lubricants, coating agents, pigments, diluents, bases, solubilizers or solubilization aids, tonicity agents, pH adjusters, stabilizers, propellants, and adhesives.

Examples of preparations suitable for the oral administration of the pharmaceutical composition comprising the compound of the present invention or a pharmaceutically acceptable salt thereof, or a solvate thereof as an active ingredient can include tablets, powders, granules, capsules, solutions, syrups, elixirs, and oily or aqueous suspensions. Moreover, examples of preparations suitable for parenteral administration can include injections, drops, suppositories, inhalants, and patches.

The dose of the pharmaceutical composition comprising the compound of the present invention or a pharmaceutically acceptable salt thereof, or a solvate thereof as an active ingredient is not particularly limited, and can be selected appropriately according to various conditions such as the age, body weight, and conditions of a patient. It is preferred to administer the pharmaceutical composition of the present invention at a dose of 1 mg to 1000 mg, preferably 5 mg to 500 mg, more preferably 5 mg to 300 mg, even more preferably 5 mg to 100 mg of the active ingredient per day in adult human, which is administered in one portion or in several portions, preferably in one portion or in two portions, per day, according to the conditions.

Specific embodiments of the present disclosure are set forth below:

(1) A stereoisomer of N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (II):

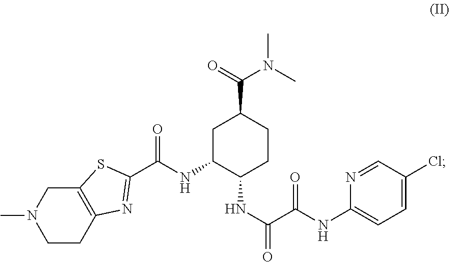

wherein the stereoisomer is selected from the group consisting of:

(a) N¹-(5-chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (III):

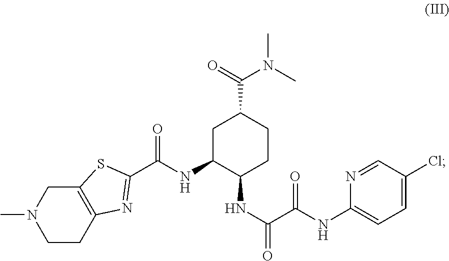

(b) N¹-(5-chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IV):

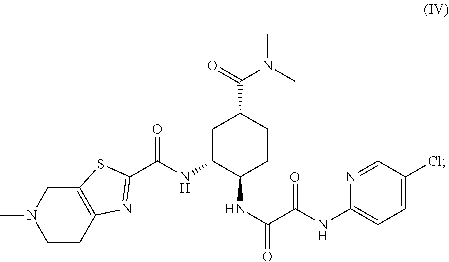

(c) N¹-(5-chloropyridin-2-yl)-N²-((1R,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (V):

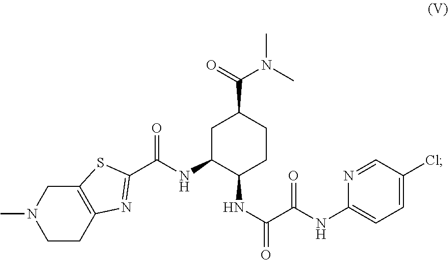

(d) N¹-(5-chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VI):

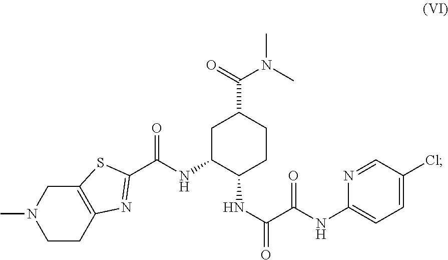

(e) N¹-(5-chloropyridin-2-yl)-N²-((1S,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VII):

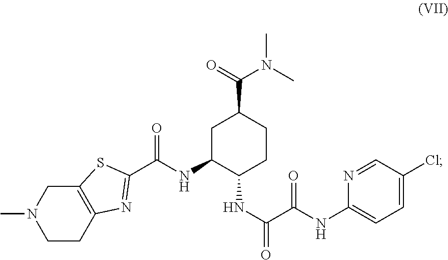

(f) N¹-(5-chloropyridin-2-yl)-N²-((1S,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VIII):

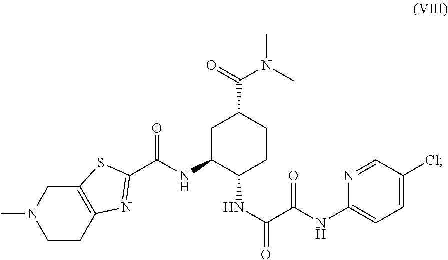

or a salt thereof, or a solvate thereof.

(2) The stereoisomer according to (1), wherein the stereoisomer is:

N¹-(5-chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (III):

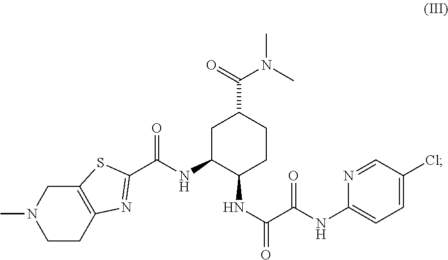

or a salt thereof, or a solvate thereof.

(3) The stereoisomer according to (1), wherein the stereoisomer is:

N¹-(5-chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IV):

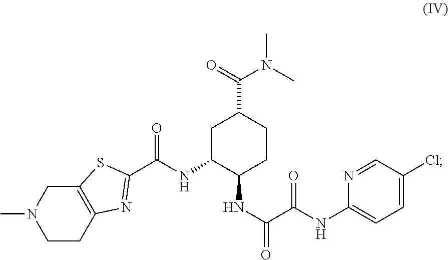

or a salt thereof, or a solvate thereof.

(4) The stereoisomer according to (1), wherein the stereoisomer is:

N¹-(5-chloropyridin-2-yl)-N²-((1R,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (V):

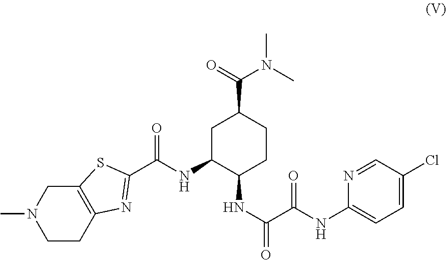

or a salt thereof, or a solvate thereof.

(5) The stereoisomer according to (1), wherein the stereoisomer is:

N¹-(5-chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VI):

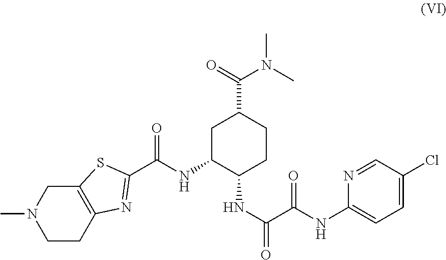

or a salt thereof, or a solvate thereof.

(6) The stereoisomer according to (1), wherein the stereoisomer is:

N¹-(5-chloropyridin-2-yl)-N²-((1S,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VII):

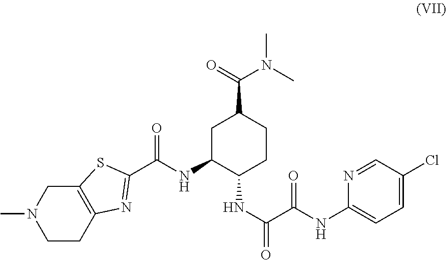

or a salt thereof, or a solvate thereof.

(7) The stereoisomer according to (1), wherein the stereoisomer is:

N¹-(5-chloropyridin-2-yl)-N²-((1S,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VIII):

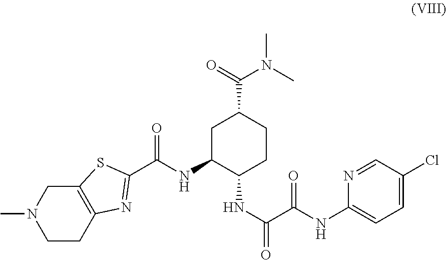

or a salt thereof, or a solvate thereof.

(8) A method for inspecting impurities in a pharmaceutical composition containing N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (II):

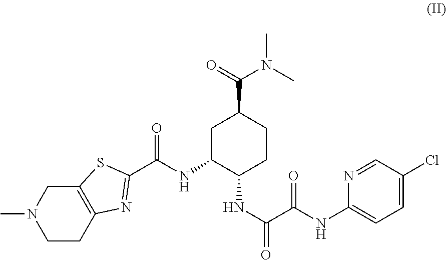

or a salt thereof, or a solvate thereof, comprising

using, as a standard, at least one or more compound(s), salt(s), or solvate(s) selected from the group consisting of:

(a) N¹-(5-chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (III):

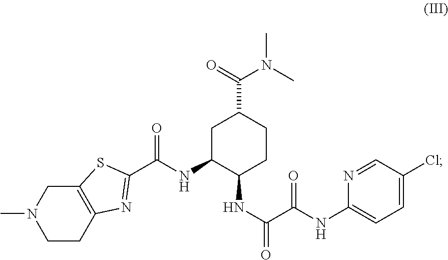

or a salt thereof, or a solvate thereof,

(b) N¹-(5-chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IV):

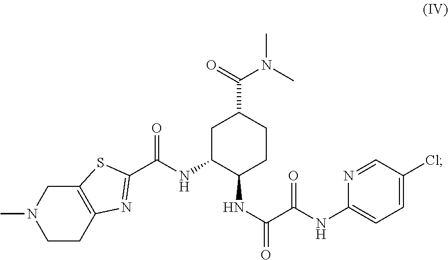

or a salt thereof, or a solvate thereof,

(c) N¹-(5-chloropyridin-2-yl)-N²-((1R,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (V):

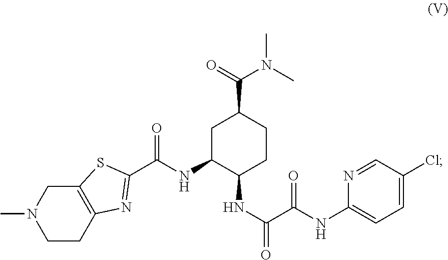

or a salt thereof, or a solvate thereof,

(d) N¹-(5-chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VI):

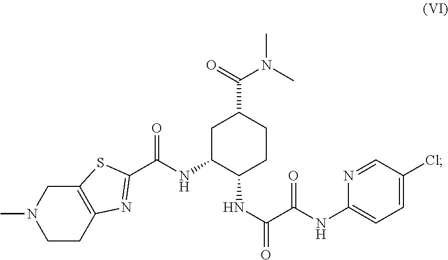

or a salt thereof, or a solvate thereof,

(e) N¹-(5-chloropyridin-2-yl)-N²-((1S,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VII):

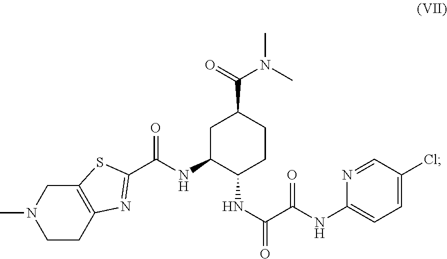

or a salt thereof, or a solvate thereof,

(f) N¹-(5-chloropyridin-2-yl)-N²-((1S,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VIII):

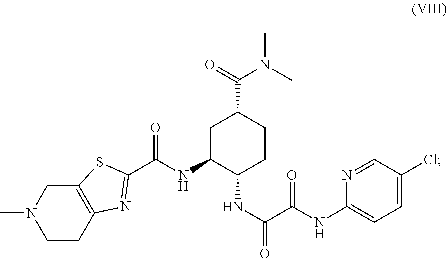

or a salt thereof, or a solvate thereof,

(g) N¹-(5-chloropyridin-2-yl)-N²-((1R,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IX):

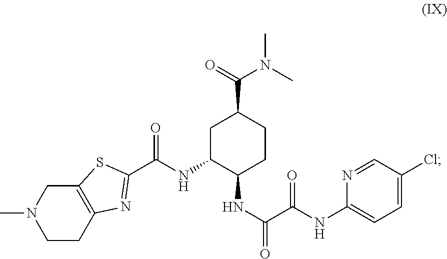

or a salt thereof, or a solvate thereof.

(9) The method according to (8), wherein the standard is:

N¹-(5-chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (III):

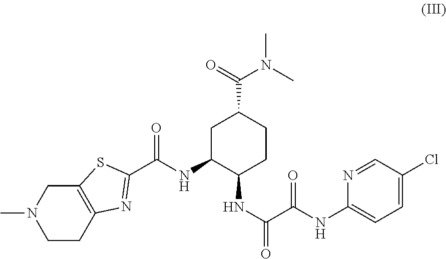

or a salt thereof, or a solvate thereof.

(10) The method according to (8), wherein the standard is:

N¹-(5-chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IV):

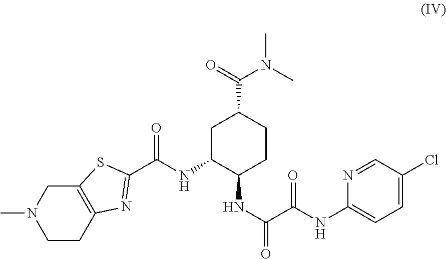

or a salt thereof, or a solvate thereof.

(11) The method according to (8), wherein the standard is:

N¹-(5-chloropyridin-2-yl)-N²-((1R,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (V):

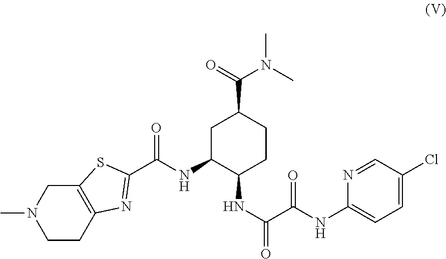

or a salt thereof, or a solvate thereof.

(12) The method according to (8), wherein the standard is:

N¹-(5-chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VI):

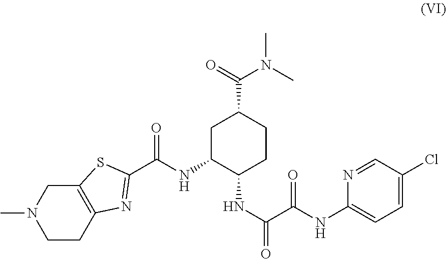

or a salt thereof, or a solvate thereof.

(13) The method according to (8), wherein the standard is:

N¹-(5-chloropyridin-2-yl)-N²-((1S,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VII):

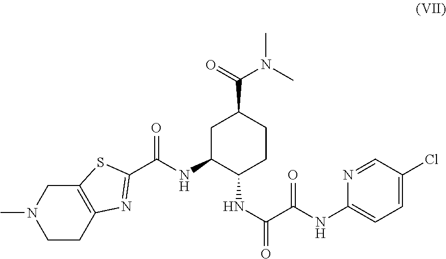

or a salt thereof, or a solvate thereof.

(14) The method according to (8), wherein the standard is:

N¹-(5-chloropyridin-2-yl)-N²-((1S,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VIII):

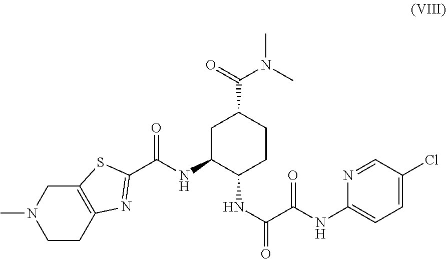

or a salt thereof, or a solvate thereof.

(15) A substantially pure N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (II):

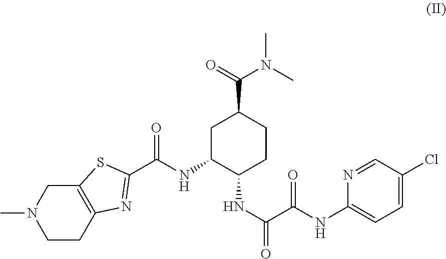

or a salt thereof, or a solvate thereof, wherein the compound II or salt, or solvate thereof is substantially free from a stereoisomer of compound II or salt thereof, or solvate thereof.

(16) The compound or a salt thereof, or a solvate thereof according to (15), wherein the content of the stereoisomer or salt thereof, or solvate thereof is 3% or less.

(17) A substantially pure pharmaceutical composition containing N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (II):

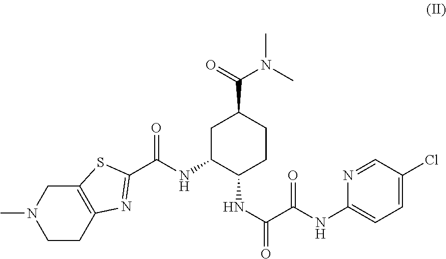

or a salt thereof, or a solvate thereof, wherein the composition is substantially free from stereoisomer of compound II or salt thereof, or solvate thereof.

(18) The pharmaceutical composition according to (17), wherein the content of the stereoisomer or salt thereof, or solvate thereof is 3% or less.

### EXAMPLES

### Example 1

### N¹-(5-Chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide (compound III)

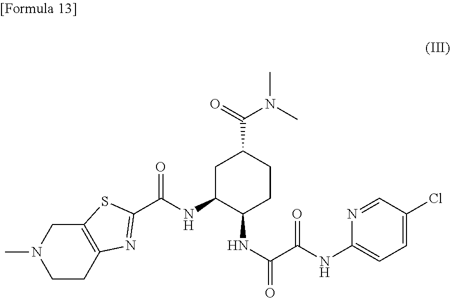

### (1-1) (1R,4R,5R)-4-Iodo-6-oxabicyclo[3.2.1]octan-7-one

A salt of (1R)-3-cyclohexene-1-carboxylic acid and (1S)-1-phenyl-1-ethanamine (36.0 g, 146 mmol) was separated into aqueous and organic layers by the addition of 2 N hydrochloric acid (500 ml) and diethyl ether (300 ml), and the aqueous layer was subjected to extraction with diethyl ether (2×250 ml). The organic layer was washed with saturated saline (100 ml) and then dried over anhydrous sodium sulfate, and the solvent was distilled off under reduced pressure. The obtained clear colorless oil (18.9 g), potassium iodide (commercially available) (31.5 g, 190 mmol), and sodium bicarbonate (commercially available) (18.4 g, 219 mmol) were dissolved in methylene chloride (200 ml) and water (200 ml). To the solution, iodine (commercially available) (48.2 g, 190 mmol) was added at 0° C. After stirring at room temperature for 2 hours, the mixture was separated into aqueous and organic layers by the addition of a 1 N aqueous sodium thiosulfate solution (300 ml), and the aqueous layer was subjected to extraction with methylene chloride (2×100 ml). The organic layers were combined, washed with water (200 ml) and saturated saline (100 ml), and then dried over anhydrous sodium sulfate, and the solvent was distilled off under reduced pressure. The obtained pale yellow solid was dissolved in methylene chloride. To the solution, hexane was then added, and a toluene-water mixed solvent (1:2) (26 ml) was added to the resulting white solid (26.8 g). After stirring for 3 days, a toluene-water mixed solvent (1:2) (52 ml) was added again to the obtained solid (26.3 g). After stirring overnight, the solid was collected by filtration and dried to obtain the title compound (97.0% ee) (25.2 g, 100 mmol, 68%) as a white solid.

¹H-NMR (400 MHz, CDCl₃) δ: 1.78-1.96 (2H, m), 2.12 (1H, dd, J=16.4, 5.1 Hz), 2.34-2.51 (2H, m), 2.68 (1H, t, J=1.9 Hz), 2.80 (1H, d, J=12.5 Hz), 4.51 (1H, t, J=4.4 Hz), 4.83 (1H, t, J=5.0 Hz).

[α]_{D}²⁵: +40.5° (c=1.0, CHCl₃).

### (1-2) (1R,3S,4S)-3-[(Tert-butoxycarbonyl)amino]-4-hydroxycyclohexanecarboxylic acid ethyl ester

(1R,4R,5R)-4-Iodo-6-oxabicyclo[3.2.1]octan-7-one (25.1 g, 99.6 mmol) was dissolved in ethanol (125 ml). To the solution, potassium carbonate (commercially available) (16.5 g, 119 mmol) was added at 90° C. The mixture was heated to reflux for 2 hours and then allowed to cool to room temperature. The precipitate was filtered off, and the filtrate was concentrated under reduced pressure. The obtained residue was separated into aqueous and organic layers by the addition of ethyl acetate (200 ml) and water (50 ml). Then, the organic layer was washed with water (50 ml) and dried over anhydrous sodium sulfate, and the solvent was distilled off under reduced pressure. The obtained pale yellow oil (16.7 g) was dissolved in N,N-dimethylformamide (120 ml). To the solution, ammonium chloride (commercially available) (7.77 g, 145 mmol) and sodium azide (commercially available) (9.45 g, 145 mmol) were added at room temperature. The mixture was stirred at 80° C. for 2 hours, then allowed to cool to room temperature, and completely poured to a stirred mixed solution of ethyl acetate (500 ml) and ice water (800 ml). After separation into aqueous and organic layers, the aqueous layer was subjected to extraction with ethyl acetate (2×200 ml), and the organic layers were then combined, washed with water (500 ml) and saturated saline (200 ml), and dried over anhydrous sodium sulfate. To the obtained ethyl acetate solution, di-tert-butyl dicarbonate (commercially available) (27.5 g, 126 mmol) and 10% palladium carbon (commercially available) (1.50 g) were added, and the mixture was stirred overnight under the hydrogen atmosphere. Insoluble matter was filtered off, and the filtrate was then concentrated under reduced pressure. The obtained residue was purified using silica gel column chromatography (silica gel: 400 g, hexane:ethyl acetate=3:1→1:1). Then, the title compound (19.3 g, 67.2 mmol, 67%) was obtained as a white solid by reprecipitation from a hexane-methylene chloride system.

¹H-NMR (400 MHz, CDCl₃) δ: 1.27 (3H, t, J=7.2 Hz), 1.39-1.60 (3H, m), 1.45 (9H, s), 1.85-1.93 (1H, m), 2.08-2.16 (1H, m), 2.29-2.37 (1H, m), 2.61-2.67 (1H, m), 3.31 (1H, br s), 3.39-3.49 (1H, m), 3.54-3.66 (1H, m), 4.10-4.24 (2H, m), 4.54 (1H, br d, J=6.1 Hz).

[α]_{D}²⁵: −26.8° (c=1.0, CHCl₃).

### (1-3) (1R,3S,4R)-4-{[(Benzyloxy)carbonyl]amino}-3-[(tert-butoxycarbonyl)amino]cyclohexanecarboxylic acid ethyl ester

To a solution of (1R,3S,4S)-3-[(tert-butoxycarbonyl)amino]-4-hydroxycyclohexanecarboxylic acid ethyl ester (7.10 g, 24.7 mmol) in ethyl acetate (140 ml), trimethylamine hydrochloride (236 mg, 2.47 mmol) and methanesulfonyl chloride (2.87 ml, 37.1 mmol) were added at 0° C., and triethylamine (5.17 ml, 37.1 mmol) was then added dropwise. After stirring for 1 hour, the mixture was separated into aqueous and organic layers by the addition of a saturated aqueous solution of ammonium chloride (150 ml) and water (150 ml). The organic layer was washed with a saturated aqueous solution of sodium bicarbonate (150 ml) and water (150 ml) and dried over anhydrous sodium sulfate, and the solvent was then distilled off under reduced pressure. To the obtained solid, hexane (100 ml) was added, and the mixture was stirred for 1 hour. Then, the precipitate was collected by filtration and dried ((1R,3S,4S)-3-[(tert-butoxycarbonyl)amino]-4-[(methanesulfonyl)oxy]cyclohexanecarboxylic acid ethyl ester). The obtained white solid (8.51 g) was dissolved in N-methylpiperidone (85 ml). To the solution, sodium azide (3.05 g, 46.9 mmol) was added at room temperature. The mixture was stirred at 80° C. for 15 hours and then cooled to 0° C. The mixture was separated into aqueous and organic layers by the addition of ethyl acetate (100 ml) and water (100 ml). The aqueous layer was subjected to extraction with ethyl acetate (100 ml), and the organic layers were combined, washed with water (2×200 ml), then dried over anhydrous sodium sulfate, and concentrated under reduced pressure until the volume became approximately 50 ml. To this solution, ethanol (10 ml) was added, and 10% palladium carbon (1.00 g) was added at room temperature. The mixture was stirred for 40 minutes under the hydrogen atmosphere (6 atm). Insoluble matter was filtered off, and the filtrate was then concentrated under reduced pressure. The obtained residue was dissolved in tetrahydrofuran (80 ml). To the solution, a saturated aqueous solution of sodium bicarbonate (80 ml) was added at room temperature, and benzyl chlorocarbonate (3.60 ml, 25.2 mmol) in N,N-dimethylformamide (10 ml) was then added at 0° C. The mixture was stirred for 30 minutes and then separated into aqueous and organic layers by the addition of water (100 ml) and ethyl acetate (100 ml). Then, the organic layer was dried over anhydrous sodium sulfate, and the solvent was distilled off under reduced pressure. To the obtained white solid, ethyl acetate (30 ml) was added, and the mixture was dissolved by heating to 70° C. Then, hexane (120 ml) was added thereto, and the mixture was gradually allowed to cool to room temperature with stirring. Ethyl acetate (20 ml) and hexane (80 ml) were added thereto at room temperature, and the mixture was stirred for 30 minutes and then at 0° C. for 5 minutes. The resulting solid was collected by filtration and then dried to obtain the title compound (5.19 g, 12.3 mmol, 50%) as a white solid.

¹H-NMR (400 MHz, CDCl₃) δ: 1.25 (3H, t, J=7.1 Hz), 1.25-1.42 (1H, m), 1.44 (9H, s), 1.48-1.68 (1H, m), 1.75-1.89 (1H, m), 1.89-2.07 (3H, m), 2.28-2.40 (1H, m), 3.69 (1H, br s), 4.05-4.17 (1H, m), 4.13 (2H, q, J=7.1 Hz), 4.57 (1H, br s), 5.03-5.13 (2H, m), 5.26 (1H, br s), 7.26-7.38 (5H, m).

### (1-4) (1R,2S,4R)-2-[(Tert-butoxycarbonyl)amino]-4-[(dimethylamino)carbonyl]cyclohexylcarbamic acid benzyl ester

(1R,3S,4R)-4-{[(Benzyloxy)carbonyl]amino}-3-[(tert-butoxycarbonyl)amino]cyclohexanecarboxylic acid ethyl ester (2.00 g, 4.76 mmol) was dissolved in tetrahydrofuran (40 ml). To the solution, water (10 ml) and lithium hydroxide (233 mg, 9.53 mmol) were then added. After stirring for 66 hours, the mixture was separated into aqueous and organic layers by the addition of diethyl ether (40 ml) and a 10% aqueous citric acid solution (40 ml), and the aqueous layer was subjected to extraction with diethyl ether (40 ml). The organic layers were combined and dried over anhydrous sodium sulfate, and the solvent was then distilled off under reduced pressure. The obtained residue was dissolved in methylene chloride (30 ml). To the solution, dimethylamine hydrochloride (776 mg, 9.52 mmol), 1-hydroxybenzotriazole (966 mg, 7.15 mmol), 1-[3-(dimethylamino)propyl]-3-ethylcarbodiimide hydrochloride (1.37 g, 7.15 mmol), and triethylamine (663 μl, 4.76 mmol) were added at room temperature. After stirring for 7 days, the mixture was separated into aqueous and organic layers by the addition of a saturated aqueous solution of sodium bicarbonate (50 ml), and the aqueous layer was then subjected to extraction with methylene chloride (30 ml). The organic layers were combined and dried over anhydrous sodium sulfate, and the solvent was then distilled off under reduced pressure. The obtained residue was purified using silica gel column chromatography (silica gel: 100 g, methylene chloride:methanol=20:1) and then dissolved in methylene chloride (3.0 ml). To the solution, hexane (60 ml) was added, and the resulting solid was collected by filtration and dried to obtain the title compound (1.32 g, 3.15 mmol, 66%) as a white solid.

¹H-NMR (400 MHz, CDCl₃) δ: 1.24-2.10 (6H, m), 1.44 (9H, s), 2.53-2.67 (1H, m), 2.93 (3H, s), 3.03 (3H, s), 3.70 (1H, br s), 4.13 (1H, br s), 4.67 (1H, br s), 5.00-5.26 (3H, m), 7.25-7.40 (5H, m).

### (1-5) (1S,2R,5R)-2-({2-[(5-Chloropyridin-2-yl)amino]-2-oxoacetyl}amino)-5-[(dimethylamino)carbonyl]cyclohexylcarbamic acid tert-butyl ester

To a solution of (1R,2S,4R)-2-[(tert-butoxycarbonyl)amino]-4-[(dimethylamino)carbonyl]cyclohexylcarbamic acid benzyl ester (1.22 g, 2.91 mmol) in methanol (20 ml), 10% palladium carbon (300 mg) was added at room temperature, and the mixture was stirred for 5 hours under the hydrogen atmosphere (1 atm). Insoluble matter was filtered off, and the filtrate was then concentrated under reduced pressure. To the obtained residue, toluene (50 ml) was added, and the mixture was concentrated again under reduced pressure. The obtained white foamy solid and 2-[(5-chloropyridin-2-yl)amino]-2-oxoacetic acid lithium salt (850 mg, 3.79 mmol) were dissolved in N,N-dimethylformamide (10 ml). To the solution, 1-hydroxybenzotriazole (512 mg, 3.79 mmol) and 1-[3-(dimethylamino)propyl]-3-ethylcarbodiimide hydrochloride (840 mg, 4.38 mmol) were added at room temperature. After stirring at the same temperature as above for 6 days, the reaction solution was concentrated under reduced pressure. To the residue, water was added, and this mixture was subjected to extraction with dichloromethane twice. The organic layer was washed with a saturated aqueous solution of sodium bicarbonate and saturated saline in this order, dried over anhydrous sodium sulfate, and then concentrated under reduced pressure. The residue was subjected to flash column chromatography with silica gel as a carrier (SI-40C, dichloromethane:methanol=50:1→30:1), and the solvent was distilled off under reduced pressure to obtain the title compound (1.0968 g, 2.34 mmol, 80%) as a white powder.

¹H NMR (CDCl₃) δ: 1.25-1.55 (1H, m), 1.46 (9H, s), 1.65-1.89 (2H, m), 1.90-2.10 (3H, m), 2.56-2.80 (1H, br), 2.95 (3H, s), 3.06 (3H, s), 3.90-4.01 (1H, m), 4.18-4.30 (1H, m), 4.65-4.90 (0.7H, br), 5.60-5.90 (0.3H, br), 7.68 (1H, dd, J=8.8, 2.4 Hz), 7.85-8.00 (1H, br), 8.15 (1H, br d, J=8.8 Hz), 8.29 (1H, d, J=2.4 Hz), 9.72 (1H, s).

### (1-6) N¹-(5-Chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide (compound III)

To a solution of (1S,2R,5R)-2-({2-[(5-chloropyridin-2-yl)amino]-2-oxoacetyl}amino)-5-[(dimethylamino)carbonyl]cyclohexylcarbamic acid tert-butyl ester (986.0 mg, 2.11 mmol) in ethanol (30.0 ml), saturated hydrochloric acid-ethanol (10.0 ml) was added, and the mixture was stirred at room temperature for 18 hours. The reaction solution was concentrated under reduced pressure. The residue was dissolved in N,N-dimethylformamide (30 ml). To the solution, lithium 5-methyl-4,5,6,7-tetrahydro[1,3]thiazolo[5,4-c]pyridine-2-carboxylate (517.0 mg, 2.53 mmol), 1-(3-dimethylaminopropyl)-3-ethylcarbodiimide hydrochloride (485.4 mg, 2.53 mmol), 1-hydroxybenzotriazole (342.1 mg, 2.53 mmol), and triethylamine (0.292 ml, 2.11 mmol) were added, and the mixture was stirred at room temperature for 18 hours. The solvent was distilled off under reduced pressure, and the residue was separated into aqueous and organic layers by the addition of water and dichloromethane. Then, the organic layer was washed with a saturated aqueous solution of sodium bicarbonate and saturated saline in this order. The organic layer was dried over anhydrous sodium sulfate and then concentrated under reduced pressure. The residue was purified by flash column chromatography with silica gel as a carrier (SI40-B, dichloromethane:methanol=20:1→10:1), and the solvent was distilled off to obtain the title compound (712.2 mg, 1.30 mmol, 62%) as a white solid.

¹H NMR (CDCl₃) δ: 1.60-1.98 (3H, m), 2.00-2.20 (3H, m), 2.52 (3H, s), 2.78-2.92 (3H, m), 2.92-3.02 (2H, m), 2.95 (3H, s), 3.06 (3H, s), 3.69 (1H, d, J=15.5 Hz), 3.75 (1H, d, J=15.5 Hz), 4.07-4.18 (1H, m), 4.65-4.72 (1H, m), 7.42 (1H, d, J=8.6 Hz), 7.68 (1H, dd, J=8.8, 2.4 Hz), 8.05 (1H, d, J=8.1 Hz), 8.16 (1H, d, J=8.8 Hz), 8.30 (1H, d, J=2.4 Hz), 9.73 (1H, s).

MS (ESI) m/z 548 [(M+H)⁺, Cl³⁵], 550 [(M+H)⁺, Cl³⁷].

### Example 2

### N¹-(5-Chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide (compound IV)

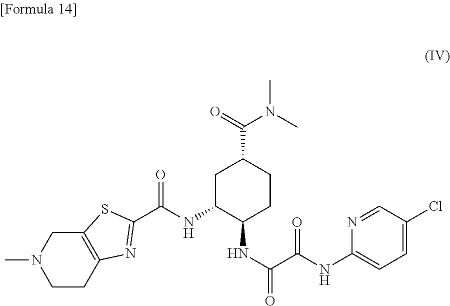

### (2-1) (1S,3R,4R)-4-{[(Benzyloxy)carbonyl]amino}-3-[(tert-butoxycarbonyl)amino]cyclohexanecarboxylic acid ethyl ester

(1S,3R,4R)-3-[(Tert-butoxycarbonyl)amino]-4-[(methanesulfonyl)oxy]cyclohexanecarboxylic acid ethyl ester (15 g) was dissolved in N,N-dimethylacetamide (150 ml). To the solution, tetrabutyl ammonium bromide (3.98 g) and sodium azide (5.35 g) were added at room temperature, and the mixture was stirred at 70° C. for 18 hours. The reaction solution was cooled to room temperature and then separated into aqueous and organic layers using ethyl acetate (225 ml) and water (225 ml), and the aqueous layer was subjected to re-extraction with ethyl acetate (75 ml). The organic layers were combined and washed with a 10% aqueous sodium bicarbonate solution (150 ml) and water (3×150 ml) in this order, and the solvent was distilled off under reduced pressure. To the obtained concentrated residue, ethanol (150 ml) and 7.5% palladium carbon (1.5 g) were added, and the mixture was stirred at 60° C. for 2.5 hours under the hydrogen atmosphere (1 atm). Insoluble matter was filtered off, and the solvent was then distilled off under reduced pressure. To the obtained concentrated residue, ethyl acetate (152 ml), water (76 ml), and sodium bicarbonate (11.2 g) were added, and benzyl chloroformate (5.0 g) was added dropwise with stirring at room temperature. Ten minutes later, the reaction mixture was separated into aqueous and organic layers, and the aqueous layer was subjected to re-extraction with ethyl acetate (38 ml). The organic layers were combined, washed with water (38 ml), and dried over anhydrous sodium sulfate, and the solvent was distilled off under reduced pressure. The obtained concentrated residue was recrystallized by the addition of ethyl acetate (20 ml) and diisopropyl ether (200 ml). The deposit was filtered off, and the solvent was then distilled off under reduced pressure to obtain a concentrated residue (8.19 g). Again, the same procedures as above were carried out for (1S,3R,4R)-3-[(tert-butoxycarbonyl)amino]-4-[(methanesulfonyl)oxy]cyclohexanecarboxylic acid ethyl ester (20 g) to obtain a concentrated residue (9.9 g). The concentrated residues were combined, crudely purified by column chromatography with silica gel as a carrier (hexane:ethyl acetate=2:1), and then repurified by column chromatography with silica gel as a carrier (hexane:ethyl acetate=5:1) to obtain the title compound (2.56 g) as a colorless oil.

¹H-NMR (CDCl₃) δ: 1.27 (3H, t, J=7.4 Hz), 1.39 (9H, s), 1.34-1.48 (2H, m), 1.50-1.59 (1H, m), 1.95-2.02 (1H, m), 2.10-2.16 (1H, m), 2.36-2.42 (1H, m), 2.71-2.76 (1H, m), 3.29-3.39 (1H, m), 3.58-3.68 (1H, m), 4.17 (2H, q, J=6.9 Hz), 4.59 (1H, d, J=8.3 Hz), 5.03-5.12 (2H, m), 5.37 (1H, d, J=6.9 Hz), 7.25-7.40 (5H, m)

### (2-2) (1R,3R,4R)-4-{[(Benzyloxy)carbonyl]amino}-3-[(tert-butoxycarbonyl)amino]cyclohexanecarboxylic acid ethyl ester

To (1S,3R,4R)-4-{[(benzyloxy)carbonyl]amino}-3-[(tert-butoxycarbonyl)amino]cyclohexanecarboxylic acid ethyl ester (1.301 g, 3.091 mmol), anhydrous ethanol (13 ml) was added under the argon atmosphere, and the mixture was dissolved by stirring at room temperature. A 20% solution of sodium ethoxide in ethanol (1.44 ml, 3.71 mmol) was added dropwise thereto with stirring under the same atmosphere as above, and the mixture was then stirred at room temperature for 3 hours and subsequently at 30° C. for 7 hours. After ice cooling, the mixture was separated into aqueous and organic layers by the addition of a 1 M aqueous sodium dihydrogen phosphate solution (26 ml) and ethyl acetate (39 ml). The aqueous layer was subjected to extraction with ethyl acetate (26 ml), and the obtained aqueous layer was subjected to re-extraction with ethyl acetate (26 ml). The organic layers were combined and washed with water (26 ml), and the obtained organic layer was washed with water (26 ml) and saturated saline (26 ml) in this order. The organic layer was dried over anhydrous sodium sulfate and then concentrated under reduced pressure. To the concentrated residue, an isopropanol-diisopropyl ether (5:95) mixed solution (6.5 ml) was added, and the mixture was stirred at 40° C. for 1 hour and then at 0° C. for 30 minutes. After filtration, the residue was washed with cold diisopropyl ether (3.5 ml) and dried under reduced pressure to obtain the title compound (0.628 g, 1.49 mmol, 48%) as a light brownish white solid.

¹H-NMR (CDCl₃) δ: 1.22 (3H, t, J=7.3 Hz), 1.20-1.29 (1H, m), 1.38 (9H, s), 1.33-1.52 (2H, m), 1.96-2.03 (1H, m), 2.12-2.18 (1H, m), 2.20-2.28 (1H, m), 2.33-2.41 (1H, m), 3.30-3.45 (2H, m), 4.10 (2H, q, J=7.3 Hz), 4.77 (1H, d, J=8.2 Hz), 5.02-5.10 (2H, m), 5.25 (1H, d, J=7.8 Hz), 7.25-7.38 (5H, m)

### (2-3) (1R,2R,4R)-2-[(Tert-butoxycarbonyl)amino]-4-[(dimethylamino)carbonyl]cyclohexylcarbamic acid benzyl ester

(1R,3R,4R)-4-{[(Benzyloxy)carbonyl]amino}-3-[(tert-butoxycarbonyl)amino]cyclohexanecarboxylic acid ethyl ester (0.200 g, 0.476 mmol), isopropanol (1.8 ml), and water (0.2 ml) were stirred at 30° C., and an aqueous lithium hydroxide solution (0.24 ml) prepared from lithium hydroxide monohydrate (0.160 g) and water (0.96 ml) was added thereto. The mixture was stirred for 2.5 hours, then adjusted to pH 7 with a 2 M aqueous hydrochloric acid solution, and concentrated under reduced pressure. To the residue, ethyl acetate (4 ml) was added, and the mixture was concentrated under reduced pressure. This procedure was repeated again. To the obtained concentrated residue, N,N-dimethylacetamide (4 ml) was added, and dimethylamine hydrochloride (0.155 g, 1.904 mmol), 1-hydroxybenzotriazole (0.096 g, 0.714 mmol), and 1-(3-dimethylaminopropyl)-3-ethylcarbodiimide hydrochloride (0.137 g, 0.714 mmol) were added in this order with stirring at room temperature. The mixture was stirred for 15.5 hours. The mixture was separated into aqueous and organic layers by the addition of ethyl acetate (4 ml) and water (4 ml), and the aqueous layer was subjected to re-extraction with ethyl acetate (3×4 ml). The organic layers were combined and washed with water (2×4 ml) and saturated saline (4 ml) in this order. The organic layer was dried over anhydrous sodium sulfate and then concentrated under reduced pressure. The residue was purified by thin-layer chromatography with silica gel as a carrier (hexane:ethyl acetate=1:3), and the solvent was distilled off to obtain the title compound (0.189 g, 0.451 mmol, 95%) as a white solid.

¹H-NMR (CDCl₃) δ: 1.18-1.35 (1H, m), 1.39 (9H, s), 1.52-1.70 (2H, m), 1.72-1.81 (1H, m), 1.98-2.07 (1H, m), 2.15-2.24 (1H, m), 2.56-2.67 (1H, m), 2.93 (3H, s), 3.03 (3H, s), 3.35-3.50 (2H, m), 4.86 (1H, d, J=7.6 Hz), 5.04-5.12 (2H, m), 5.26 (1H, d, J=6.6 Hz), 7.26-7.40 (5H, m)

### (2-4) N¹-(5-Chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide (compound IV)

(1R,2R,4R)-2-[(Tert-butoxycarbonyl)amino]-4-[(dimethylamino)carbonyl]cyclohexylcarbamic acid benzyl ester (0.189 g, 0.451 mmol) was dissolved by the addition of methanol (2 ml). To the solution, 7.5% palladium carbon (0.040 g) was then added, and the mixture was stirred at room temperature for 4.5 hours under the hydrogen atmosphere (1 atm). Insoluble matter was filtered off, and the filtrate was then concentrated under reduced pressure. To the residue, ethyl acetate (4 ml) was added, and the mixture was concentrated under reduced pressure. This procedure was repeated again. The obtained concentrated residue was dissolved by the addition of N,N-dimethylacetamide (2 ml). To the solution, 2-[(5-chloropyridin-2-yl)amino]-2-oxoacetic acid lithium salt (0.103 g, 0.497 mmol), 1-hydroxybenzotriazole (0.073 g, 0.542 mmol), and 1-(3-dimethylaminopropyl)-3-ethylcarbodiimide hydrochloride (0.104 g, 0.542 mmol) were then added in this order, and the mixture was stirred at room temperature for 17 hours. Further, 1-(3-dimethylaminopropyl)-3-ethylcarbodiimide hydrochloride (0.010 g, 0.052 mmol) was added thereto. After stirring at room temperature for 4.5 hours, water (2.5 mL) was added thereto, and the mixture was suspended and stirred for 30 minutes and then filtered. The residue was washed with water (4.5 ml) and then dried under reduced pressure to obtain a white solid. Moreover, the filtrate was subjected to extraction with ethyl acetate (3×7 ml), and the organic layers were combined and washed with water (2×5 ml) and saturated saline (3 ml) in this order. The organic layer was dried over anhydrous sodium sulfate and then concentrated under reduced pressure to obtain a light brown oil. This light brown oil and the preceding obtained white solid were combined. Acetonitrile (3.2 ml) was added thereto, and the mixture was suspended and stirred. Methanesulfonic acid (0.112 ml, 1.72 mmol) was added thereto. After stirring at room temperature for 6 hours, triethylamine (0.262 ml, 1.892 mmol) was added thereto at 0° C. 5-Methyl-4,5,6,7-tetrahydro[1,3]thiazolo[5,4-c]pyridine-2-carboxylic acid hydrochloride (0.089 g, 0.378 mmol), 1-hydroxybenzotriazole (0.056 g, 0.413 mmol), and 1-(3-dimethylaminopropyl)-3-ethylcarbodiimide hydrochloride (0.079 g, 0.413 mmol) were added thereto in this order at room temperature. After stirring at room temperature for 3 hours, the mixture was adjusted to pH 8 to 9 by the addition of triethylamine (0.072 ml, 0.52 mmol), and water (3.2 ml) was added thereto. After stirring overnight at room temperature, the mixture was separated into aqueous and organic layers by the addition of chloroform (4.8 ml). The aqueous layer was subjected to re-extraction by the addition of chloroform (2×3.2 ml), and the organic layers were combined and washed with water (2×1.6 mL) and saturated saline (1.6 mL) in this order. The organic layer was dried over anhydrous sodium sulfate and then concentrated under reduced pressure. The residue was purified by thin-layer chromatography with silica gel as a carrier (chloroform:methanol=19:1), and the solvent was distilled off to obtain the title compound (0.146 g, 0.266 mmol) as a yellow solid.

¹H-NMR (CDCl₃) δ: 1.48-1.57 (1H, m), 1.69-1.79 (1H, m), 1.83-1.92 (2H, m), 2.13-2.19 (1H, m), 2.25-2.31 (1H, m), 2.47 (3H, s), 2.72-2.85 (3H, m), 2.85-2.90 (2H, m), 2.95 (3H, s), 3.07 (3H, s), 3.60 (1H, d, J=15.6 Hz), 3.69 (1H, d, J=15.6 Hz), 3.85-3.93 (1H, m), 4.12-4.20 (1H, m), 7.46 (1H, d, J=8.7 Hz), 7.67 (1H, dd, J=2.3, 8.7 Hz), 7.97 (1H, d, J=8.3 Hz), 8.16 (1H, d, J=8.7 Hz), 8.27 (1H, d, J=2.3 Hz), 9.66 (1H, br)

### Example 3

### N¹-(5-Chloropyridin-2-yl)-N²-((1R,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide (compound V)

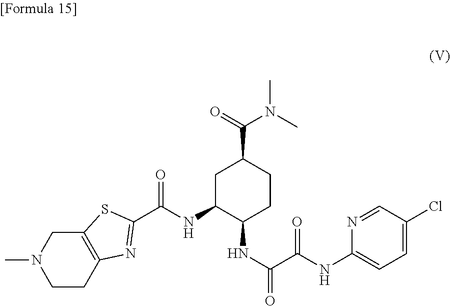

A (R)-(+)-α-methylbenzylamine salt of (1S)-3-cyclohexene-1-carboxylic acid (J. Am. Chem. Soc., 1978, Vol. 100, p. 5199-5203) (49.5 g, 0.2 mol) was separated into aqueous and organic layers by the addition of ethyl acetate and 4 N hydrochloric acid, and the aqueous layer was subjected to re-extraction with ethyl acetate twice. The organic layers were combined, washed with saturated saline, and dried over anhydrous magnesium sulfate, and the solvent was distilled off under reduced pressure. The obtained residue was dissolved in N,N-dimethylformamide (275 ml). To the solution, triethylamine (85 ml, 0.61 mol) and benzyl bromide (31 ml, 0.26 mol) were added under ice cooling, and the mixture was stirred at room temperature for 24 hours. After addition of ethyl acetate and water, the aqueous layer was subjected to extraction with ethyl acetate three times. The combined organic layer was washed with saturated saline and then dried over anhydrous magnesium sulfate, and the solvent was distilled off under reduced pressure. The obtained residue was purified by silica gel chromatography (hexane:ethyl acetate=3:1) to obtain (S)-3-cyclohexene-1-carboxylic acid benzyl ester (14.2 g) as a red-brown oil.

This (S)-3-cyclohexene-1-carboxylic acid benzyl ester (10 g) was dissolved in dichloromethane (125 ml). To the solution, m-chloroperbenzoic acid (11.2 g, 64.7 mmol) was added under ice cooling, and the mixture was stirred for 2 hours. A 10% aqueous sodium thiosulfate solution was added thereto, and the mixture was stirred for 20 minutes. Then, the separated aqueous layer was subjected to extraction with dichloromethane three times. The combined organic layer was washed with a saturated aqueous solution of sodium bicarbonate and saturated saline in this order and dried over anhydrous magnesium sulfate, and the solvent was distilled off under reduced pressure. The obtained residue was purified by silica gel column chromatography (ethyl acetate:hexane=9:1) to obtain (1R,3S,6S)-7-oxabicyclo[4.1.0]heptane-3-carboxylic acid benzyl ester (5.2 g) as a colorless oil.

This (1R,3S,6S)-7-oxabicyclo[4.1.0]heptane-3-carboxylic acid benzyl ester (5.2 g) was dissolved in N,N-dimethylformamide (100 ml). To the solution, ammonium chloride (2.16 g, 40 mmol) and sodium azide (1.82 g, 28 mmol) were added, and the mixture was stirred at 70° C. Twenty-four hours later, the solvent was distilled off under reduced pressure, and the obtained residue was separated into aqueous and organic layers by the addition of ethyl acetate and water. The organic layer was washed with saturated saline. The organic layer was dried over anhydrous magnesium sulfate, and the solvent was then distilled off under reduced pressure to obtain a colorless oil (6.1 g).

This colorless oil (6 g) was dissolved in tetrahydrofuran (30 ml). To the solution, triphenylphosphine (6.6 g, 24 mmol) and water (0.59 ml, 33 mmol) were added, and the mixture was stirred at room temperature. Twenty hours later, di-tert-butyl dicarbonate (5.4 g, 25 mmol) was added thereto, and the mixture was further stirred for 2 hours.

The solvent was distilled off under reduced pressure, and the residue was purified by silica gel column chromatography (hexane:ethyl acetate=2:1) to obtain (1S,3R,4R)-4-[(tert-butoxycarbonyl)amino]-3-hydroxycyclohexanecarboxylic acid benzyl ester (6.5 g) as a colorless oil.

This (1S,3R,4R)-4-[(tert-butoxycarbonyl)amino]-3-hydroxycyclohexanecarboxylic acid benzyl ester (6.5 g) was dissolved in ethyl acetate (100 ml). To the solution, 10% palladium carbon (0.65 g) was added, and the mixture was stirred at room temperature for 20 hours under hydrogen stream. Insoluble matter was filtered, and the solvent was then distilled off under reduced pressure. A portion (1.9 g) of the obtained concentrated residue was dissolved in a mixed solvent of methanol (8 ml) and toluene (15 ml). To the solution, 2 M trimethylsilyldiazomethane was added under ice cooling until yellow color persisted. The mixture was stirred at room temperature for 30 minutes. The solvent was distilled off under reduced pressure, and the residue was purified by silica gel column chromatography (hexane:ethyl acetate=1:1) to obtain (1S,3R,4R)-4-[(tert-butoxycarbonyl)amino]-3-hydroxycyclohexanecarboxylic acid methyl ester (2 g) as a colorless oil.

This (1S,3R,4R)-4-[(tert-butoxycarbonyl)amino]-3-hydroxycyclohexanecarboxylic acid methyl ester (2 g) was dissolved in dichloromethane (46 ml). To the solution, triethylamine (5.85 ml, 42 mmol) and methanesulfonyl chloride (1.96 ml, 25.2 mmol) were added under ice cooling, and the mixture was stirred for 1 hour. Dichloromethane and 1 N hydrochloric acid were added thereto, and the mixture was stirred. Then, the aqueous layer was subjected to extraction with dichloromethane three times, and the combined organic layer was washed with saturated saline. The organic layer was dried over anhydrous magnesium sulfate, and the solvent was then distilled off under reduced pressure to obtain a pale yellow oil.

This pale yellow oil was dissolved in N-methylpyrrolidone (6 ml). To the solution, sodium azide (1.1 g, 17 mmol) and tetrabutyl ammonium chloride (0.71 g, 2.55 mmol) were added, and the mixture was stirred at 70° C. for 12 hours. The mixture was separated into aqueous and organic layers by the addition of ethyl acetate and water. The aqueous layer was subjected to extraction with ethyl acetate three times, and the combined organic layer was washed with saturated saline. The organic layer was dried over anhydrous magnesium sulfate, and the solvent was then distilled off under reduced pressure. The obtained residue was purified by silica gel column chromatography (ethyl acetate:hexane=1:4) to obtain a mixture of (1S,3S,4R)-3-azido-4-[(tert-butoxycarbonyl)amino]cyclohexanecarboxylic acid methyl ester and (1S,3R,4R)-3-azido-4-[(tert-butoxycarbonyl)amino]cyclohexanecarboxylic acid methyl ester as a colorless oil.

This mixture was dissolved in ethyl acetate (80 ml). To the solution, 10% palladium carbon (0.23 g) was added, and the mixture was stirred for 20 hours under hydrogen stream. Insoluble matter was filtered, and the solvent was then distilled off under reduced pressure to obtain a pale yellow oil (0.91 g) as a residue.

To this residue (0.5 g), ethyl acetate (10 ml) and water (5 ml) were added, and sodium bicarbonate (0.75 g) was added. To the mixture, benzyl chloroformate (0.51 ml) was then added dropwise. After the completion of reaction, the mixture was separated into aqueous and organic layers by the addition of ethyl acetate, and the organic layer was washed with saturated saline and dried over anhydrous magnesium sulfate. Insoluble matter was filtered, and the solvent was then distilled off under reduced pressure. The obtained residue was purified by silica gel column chromatography (ethyl acetate:hexane=1:4) to obtain a mixture of (1S,3S,4R)-3-{[(benzyloxy)carbonyl]amino}-4-[(tert-butoxycarbonyl)amino]cyclohexanecarboxylic acid methyl ester and (1S,3R,4R)-3-{[(benzyloxy)carbonyl]amino}-4-[(tert-butoxycarbonyl)amino]cyclohexanecarboxylic acid methyl ester (0.6 g) as a colorless oil.

To this mixture (0.200 g, 0.492 mmol), isopropanol (1.8 ml) and water (0.2 ml) were added, and lithium hydroxide monohydrate (0.040 g) and water (0.48 ml) were added. The mixture was stirred at room temperature for 6 hours, then adjusted to pH 7 to 8 with 2 N hydrochloric acid (0.04 ml), and concentrated under reduced pressure. To the residue, ethyl acetate (2 ml) was added, and the mixture was concentrated again under reduced pressure. This procedure was repeated again. To the obtained concentrated residue, N,N-dimethylacetamide (2 ml) was added, and dimethylamine hydrochloride (0.161 g, 1.968 mmol), 1-hydroxybenzotriazole (0.100 g, 0.738 mmol), and 1-(3-dimethylaminopropyl)-3-ethylcarbodiimide hydrochloride (0.141 g, 0.738 mmol) were added in this order with stirring at room temperature. After stirring overnight, the mixture was separated into aqueous and organic layers by the addition of ethyl acetate (2 ml) and water (2 ml), and the aqueous layer was subjected to re-extraction with ethyl acetate (2×2 ml). The organic layers were combined, washed with water (1 ml) and saturated saline (1 ml) in this order, and dried over anhydrous sodium sulfate, and the solvent was distilled off under reduced pressure. The obtained residue was purified by thin-layer chromatography with silica gel as a carrier (hexane:ethyl acetate=1:3) to obtain a mixture of (1R,2S,4S)-2-{[(benzyloxy)carbonyl]amino}-4-[(dimethylamino)carbonyl]cyclohexylcarbamic acid tert-butyl ester and (1R,2R,4S)-2-{[(benzyloxy)carbonyl]amino}-4-[(dimethylamino)carbonyl]cyclohexylcarbamic acid tert-butyl ester (0.157 g) as a clear colorless oil.

To this mixture, isopropanol (3 ml) was added, and 7.5% palladium carbon (0.031 g) was added. The mixture was stirred at 35° C. under the hydrogen atmosphere. 21 hours later, 7.5% palladium carbon (0.015 g) was added thereto, and the mixture was stirred at the same temperature under the same atmosphere as above. Eight hours later, insoluble matter was filtered off, and the filtrate was concentrated under reduced pressure. To the obtained concentrated residue, N,N-dimethylacetamide (1 ml) was added, and triethylamine (0.077 ml, 0.557 mmol), 5-methyl-4,5,6,7-tetrahydro[1,3]thiazolo[5,4-c]pyridine-2-carboxylic acid hydrochloride (0.096 g, 0.408 mmol), 1-hydroxybenzotriazole (0.060 g, 0.445 mmol), and 1-(3-dimethylaminopropyl)-3-ethylcarbodiimide hydrochloride (0.085 g, 0.445 mmol) were added in this order at room temperature. After stirring for 4 hours, the mixture was cooled on ice and adjusted to pH 8 to 9 by the addition of water (1 ml) and triethylamine (0.072 ml). The mixture was separated into aqueous and organic layers by the addition of ethyl acetate (2 ml) and water (1 ml) thereto, and the aqueous layer was then subjected to re-extraction with ethyl acetate (2×2 ml). The organic layers were combined, washed with water (2×1 ml) and saturated saline (1 ml) in this order, dried over anhydrous sodium sulfate, and concentrated under reduced pressure.

To the obtained concentrated residue, acetonitrile (3.4 ml) was added, and methanesulfonic acid (0.048 ml, 0.734 mmol) was added at room temperature. The mixture was stirred. 2 hours later, methanesulfonic acid (0.048 ml) was added thereto, and further 3 hours later, methanesulfonic acid (0.024 ml) was added thereto. After stirring for 2 hours, the mixture was adjusted to pH 8 to 9 by the addition of triethylamine (0.254 ml, 1.835 mmol) in an ice bath. To this reaction solution, 2-[(5-chloropyridin-2-yl)amino]-2-oxoacetic acid lithium salt (0.083 g, 0.404 mmol), 1-hydroxybenzotriazole (0.059 g, 0.440 mmol), and 1-(3-dimethylaminopropyl)-3-ethylcarbodiimide hydrochloride (0.084 g, 0.440 mmol) were added in this order at room temperature, and the mixture was stirred for 3.5 hours. After further stirring at 30° C. for 21 hours, the mixture was adjusted to approximately pH 9 by the addition of triethylamine (0.075 ml), and the solvent was then distilled off under reduced pressure. The obtained concentrated residue was separated into aqueous and organic layers by the addition of chloroform (4 ml) and water (2 ml), and the aqueous layer was then subjected to re-extraction by the addition of chloroform (5×2 ml). The organic layers were combined, washed with water (1 ml), and dried over anhydrous sodium sulfate, and the solvent was distilled off under reduced pressure. The obtained concentrated residue was purified by thin-layer chromatography with silica gel as a carrier (chloroform:methanol=15:1), and the solvent was distilled off to obtain the title compound (0.064 g) and N¹-(5-chloropyridin-2-yl)-N²-((1R,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide (0.033 g).

¹H-NMR (THF-d8) δ: 1.63-1.68 (2H, m), 1.70-1.78 (1H, m), 1.78-1.84 (1H, m), 2.08-2.15 (1H, m), 2.21-2.29 (1H, m), 2.40 (3H, s), 2.66-2.76 (2H, m), 2.76-2.80 (2H, m), 2.89 (3H, s), 2.90-3.00 (1H, m), 3.08 (3H, s), 3.56-3.64 (2H, m), 4.30-4.35 (1H, m), 4.36-4.42 (1H, m), 7.80 (1H, dd, J=2.8, 9.2 Hz), 8.20 (1H, d, J=9.6 Hz), 8.30 (1H, d, J=2.8 Hz), 8.27-8.31 (1H, m), 8.45 (1H, d, J=8.3 Hz), 10.0 (1H, br)

### Example 4

### N¹-(5-Chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide (compound VI)

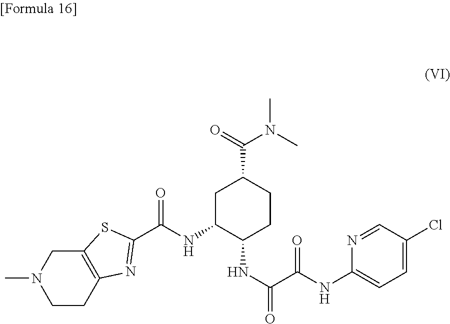

### (4-1) (1R,3R,4S)-4-{[(Benzyloxy)carbonyl]amino}-3-[(tert-butoxycarbonyl)amino]cyclohexanecarboxylic acid ethyl ester

To (1S,3R,4S)-4-{[(benzyloxy)carbonyl]amino}-3-[(tert-butoxycarbonyl)amino]cyclohexanecarboxylic acid ethyl ester (1.300 g, 3.091 mmol), anhydrous tetrahydrofuran (9.1 ml) and anhydrous ethanol (3.9 ml) were added under the nitrogen atmosphere, and the mixture was dissolved by stirring at room temperature. To the solution, a 20% solution of sodium ethoxide in ethanol (1.44 ml, 3.71 mmol) was added dropwise. After stirring at 30° C. for 4 hours, the mixture was cooled on ice and separated into aqueous and organic layers by the addition of 1 M sodium dihydrogen phosphate (26 mL) and ethyl acetate (39 mL), and the aqueous layer was subjected to re-extraction with ethyl acetate (2×26 ml). The organic layers were combined, washed with water (2×26 ml) and saturated saline (26 ml) in this order, and dried over anhydrous sodium sulfate, and the solvent was distilled off under reduced pressure. To the obtained concentrated residue, diisopropyl ether (3.9 ml) was added, and the mixture was suspended and stirred at 40° C. for 30 minutes. After stirring for 30 minutes under ice cooling, the solid was collected by filtration. This solid was purified by thin-layer chromatography with silica gel as a carrier (chloroform:hexane=9:1), and the solvent was distilled off to obtain the title compound (0.094 g) as a white solid.

¹H-NMR (CDCl₃) δ: 1.25 (3H, t, J=7.3 Hz), 1.45 (9H, s), 1.38-1.56 (2H, m), 1.62-1.72 (1H, m), 1.77-1.84 (1H, m), 1.84-1.90 (1H, m), 2.06-2.14 (1H, m), 2.40-2.50 (1H, m), 3.66-3.73 (1H, m), 4.02-4.08 (1H, m), 4.13 (2H, q, J=7.4 Hz), 5.05-5.10 (1H, m), 5.10-5.13 (2H, m), 5.16-5.23 (1H, m), 7.30-7.40 (5H, m)

### (4-2) N¹-(5-Chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide (compound VI)

(1R,3R,4S)-4-{[(Benzyloxy)carbonyl]amino}-3-[(tert-butoxycarbonyl)amino]cyclohexanecarboxylic acid ethyl ester (10.18 g, 24.21 mmol), isopropanol (92 ml), and water (10 ml) were stirred at 30° C., and an aqueous lithium hydroxide solution (12.2 ml) prepared from lithium hydroxide monohydrate (2.50 g) and water (15 ml) was added thereto. The mixture was stirred for 3 hours, then adjusted to pH 7 with a 2 M aqueous hydrochloric acid solution, and concentrated under reduced pressure. To the residue, ethyl acetate (204 ml) was added, and the mixture was concentrated under reduced pressure. This procedure was repeated again. To the obtained concentrated residue, N,N-dimethylacetamide (204 ml) was added, and dimethylamine hydrochloride (7.89 g, 96.92 mmol), 1-hydroxybenzotriazole (4.90 g, 36.44 mmol), and 1-(3-dimethylaminopropyl)-3-ethylcarbodiimide hydrochloride (6.96 g, 36.27 mmol) were added in this order with stirring at room temperature. The mixture was stirred for 17 hours. The mixture was separated into aqueous and organic layers by the addition of ethyl acetate (610 ml) and water (459 ml), and the aqueous layer was then subjected to re-extraction with ethyl acetate (410 ml). The organic layers were combined, dried over anhydrous sodium sulfate, and then concentrated under reduced pressure to obtain (1S,2R,4R)-2-[(tert-butoxycarbonyl)amino]-4-[(dimethylamino)carbonyl]cyclohexylcarbamic acid benzyl ester (45.12 g) as a white solid.

This solid was dissolved by the addition of methanol (204 ml). To the solution, 5% palladium carbon (1.02 g) was then added, and the mixture was stirred at room temperature for 4 hours under the hydrogen atmosphere (1 atm). Insoluble matter was filtered off, and the filtrate was then concentrated under reduced pressure. To the residue, ethyl acetate (204 ml) was added, and the mixture was concentrated under reduced pressure. This procedure was repeated again. The obtained concentrated residue was dissolved by the addition of N,N-dimethylacetamide (102 ml). To the solution, 2-[(5-chloropyridin-2-yl)amino]-2-oxoacetic acid lithium salt (5.34 g, 26.6 mmol), 1-hydroxybenzotriazole (3.92 g, 29.0 mmol), and 1-(3-dimethylaminopropyl)-3-ethylcarbodiimide hydrochloride (5.56 g, 29.0 mmol) were then added in this order, and the mixture was stirred at room temperature for 16 hours. Acetonitrile (147 ml) was added thereto, and the mixture was suspended and stirred. Methanesulfonic acid (5.09 ml, 78.5 mmol) was added thereto. After stirring at room temperature for 4 hours, triethylamine (12.0 ml, 86.4 mmol) was added thereto at 0° C. 5-Methyl-4,5,6,7-tetrahydro[1,3]thiazolo[5,4-c]pyridine-2-carboxylic acid hydrochloride (4.05 g, 17.3 mmol), 1-hydroxybenzotriazole (2.55 g, 18.8 mmol), and 1-(3-dimethylaminopropyl)-3-ethylcarbodiimide hydrochloride (3.60 g, 18.8 mmol) were added thereto in this order at room temperature. After stirring at room temperature for 5 hours, triethylamine (3.28 ml, 23.6 mmol) was added thereto, and water (147 ml) was added thereto. After stirring overnight at room temperature, the mixture was separated into aqueous and organic layers by the addition of chloroform (140 ml). The aqueous layer was subjected to re-extraction by the addition of chloroform (140 ml). The organic layers were combined and washed with water (2×140 mL). The organic layer was dried over anhydrous sodium sulfate and then concentrated under reduced pressure. The residue was purified by chromatography with silica gel as a carrier, and the solvent was distilled off to obtain the title compound (8.17 g) as a yellow solid.

¹H-NMR (CDCl₃) δ: 1.81-1.88 (2H, m), 1.92-1.97 (2H, m), 2.00-2.07 (2H, m), 2.45 (3H, s), 2.75-2.82 (2H, m), 2.85-2.92 (2H, m), 2.93 (3H, s), 2.95-2.98 (1H, m), 3.09 (3H, s), 3.64-3.77 (2H, m), 4.38-4.42 (2H, m), 7.72 (1H, dd), 8.00 (1H, d), 8.21 (1H, d), 8.29-8.30 (1H, m), 8.52 (1H, d), 9.70 (1H, br)

### Example 5

### N¹-(5-Chloropyridin-2-yl)-N²-((1S,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide (compound VII)

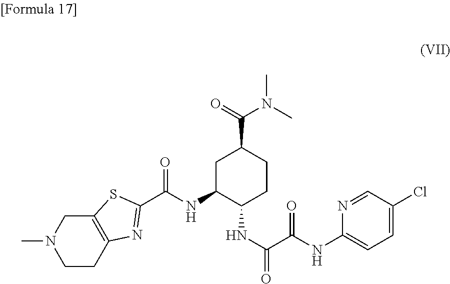

(1R,3S,4S)-3-[(Tert-butoxycarbonyl)amino]-4-[(methanesulfonyl)oxy]cyclohexanecarboxylic acid ethyl ester obtained by the method described in (1-3) of Example 1 can be treated in the same way as the method described in Example 2 to obtain the title compound.

### Example 6

### N¹-(5-Chloropyridin-2-yl)-N²-((1S,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide (compound VIII)

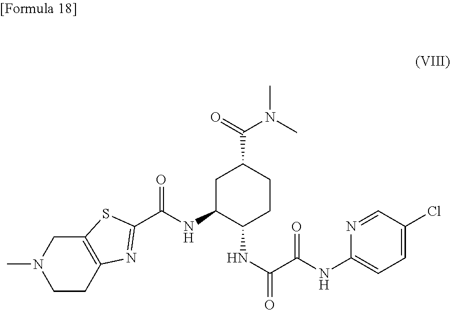

A (S)-(−)-α-methylbenzylamine salt of (1R)-3-cyclohexene-1-carboxylic acid (J. Am. Chem. Soc., 1978, Vol. 100, p. 5199-5203) can be used and treated in the same way as the method described in Reference Examples 244, 245, 246, 247, 248, 384, 385, and 387 and Example 269 described in WO 03/680 to obtain the title compound.

### Example 7

Compound II was produced according to the method described in Production Example 16 of WO 2005/047296 and Reference Example 2 of WO 2007/032498. The obtained compound II was examined for the contents of compounds III to IX as impurities using liquid chromatography.

Measurement conditions for compound III (liquid chromatography)

Detector: UV spectrophotometer (measurement wavelength: 290 nm)

Analysis column: stainless tube of 4.6 mm in internal diameter and 25 cm in length charged with silica gel carrying 10 μm amylose tris(3,5-dimethylphenylcarbamate) for liquid chromatography

Column temperature: constant temperature around 40° C.

Sample temperature: constant temperature around 25° C.

Mobile phase: ethanol

Flow rate: adjusted such that the retention time of compound II is approximately 40 minutes.

Measurement conditions for compounds IV to IX (liquid chromatography)

The amounts of compounds IV to IX were respectively measured as the total amounts of combinations between compounds in enantiomeric relationship. Specifically, the total amount of compounds IV and VII, the total amount of compounds V and VI, and the total amount of compounds VIII and IX were measured under the following conditions:

Detector: UV spectrophotometer (measurement wavelength: 290 nm)

Analysis column: stainless tube of 4.6 mm in internal diameter and 15 cm in length charged with 3 μm octadecylsilylated silica gel for liquid chromatography

Column temperature: constant temperature around 35° C.

Sample temperature: constant temperature around 10° C.

Mobile phase A: mixed solution of 0.02 mol/L phosphate buffer (pH 7.0) and acetonitrile for liquid chromatography (9:1)

Mobile phase B: mixed solution of acetonitrile for liquid chromatography and 0.02 mol/L phosphate buffer (pH 7.0) (7:3)

Flow rate: adjusted such that the retention time of compound II is approximately 16 minutes.

Solution sending of mobile phases: concentration gradient is controlled by changing the mixing ratio of mobile phases A and B as follows:

TABLE 1

| Time after injection | Mobile phase A | Mobile phase B |
|---|---|---|
| (min) | (vol %) | (vol %) |
| 0-30 | 70 | 30 |
| 30-35 | 0 | 100 |
| 35-45 | 70 | 30 |

As a result, the amount of compound III was 0.03% or less, and the total amount of compounds IV and VII, the total amount of compounds V and VI, and the total amount of compounds VIII and IX were respectively 0.01% or less.

### INDUSTRIAL APPLICABILITY

A compound of the present invention or a salt thereof, or a solvate thereof can be used as a standard in a test for inspecting impurities in pharmaceutical drugs containing compound II or a salt thereof, or a solvate thereof (e.g., compound I). A substantially pure compound II or salt thereof, or solvate thereof which is substantially free from isomer of compound II or salt thereof, or solvate thereof are useful as pharmaceuticals. A substantially pure pharmaceutical composition containing compound II or salt thereof, or solvate thereof, which is substantially free from isomer of compound II or salt thereof, or solvate thereof are useful for pharmaceuticals.

1. A stereoisomer of N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (II): 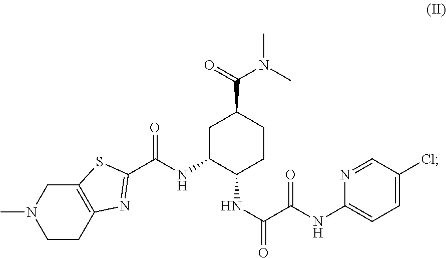
wherein the stereoisomer is selected from the group consisting of:
(a) N¹-(5-chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (III): 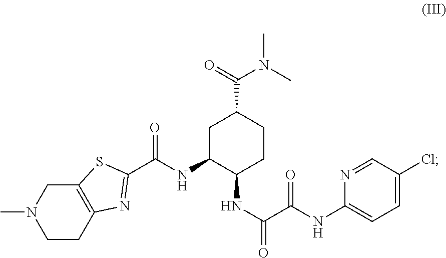
(b) N¹-(5-chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IV): 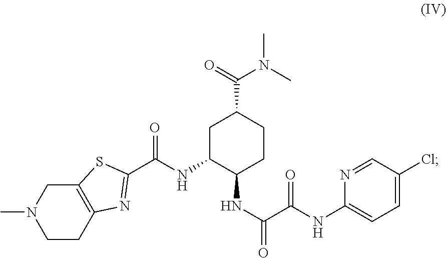
(c) N¹-(5-chloropyridin-2-yl)-N²-((1R,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (V): 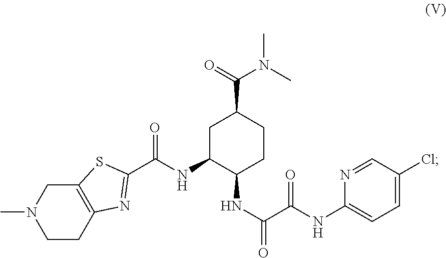
(d) N¹-(5-chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VI): 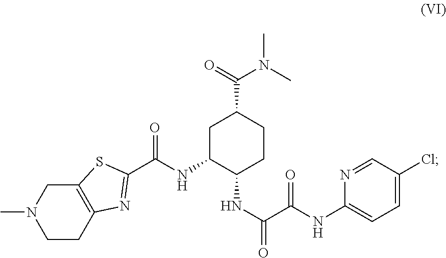
(e) N¹-(5-chloropyridin-2-yl)-N²-((1S,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VII): 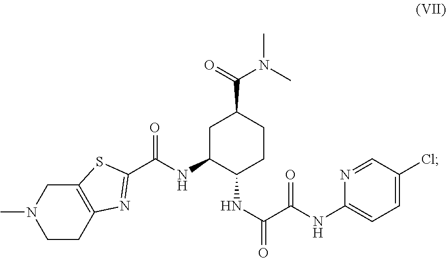
(f) N¹-(5-chloropyridin-2-yl)-N²-((1S,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VIII): 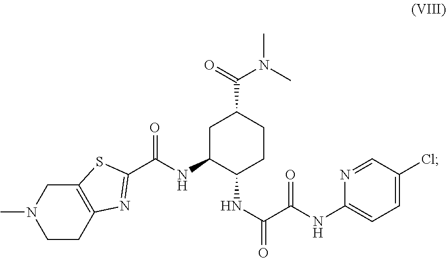
or a salt thereof, or a solvate thereof.

2. The stereoisomer according to claim 1, wherein the stereoisomer is:
N¹-(5-chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (III): 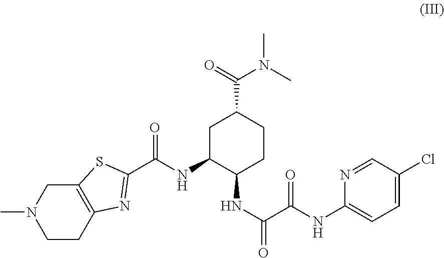
or a salt thereof, or a solvate thereof.

3. The stereoisomer according to claim 1, wherein the stereoisomer is:
N¹-(5-chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IV): 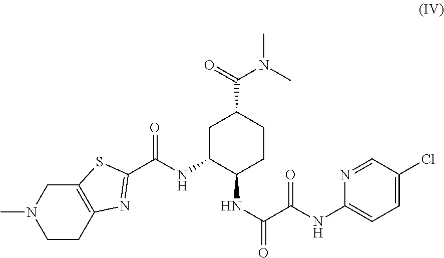
or a salt thereof, or a solvate thereof.

4. The stereoisomer according to claim 1, wherein the stereoisomer is:
N¹-(5-chloropyridin-2-yl)-N²-((1R,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (V): 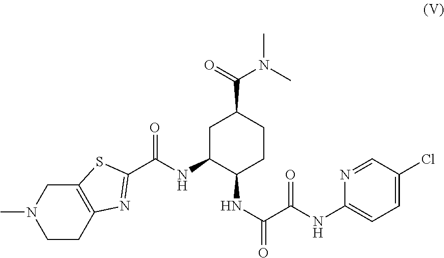
or a salt thereof, or a solvate thereof.

5. The stereoisomer according to claim 1, wherein the stereoisomer is:
N¹-(5-chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VI): 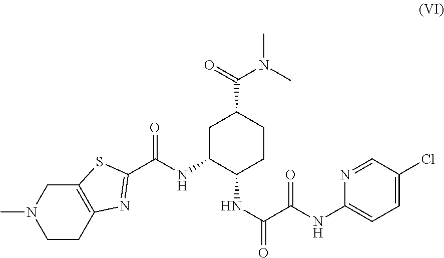
or a salt thereof, or a solvate thereof.

6. The stereoisomer according to claim 1, wherein the stereoisomer is:
N¹-(5-chloropyridin-2-yl)-N²-((1S,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VII): 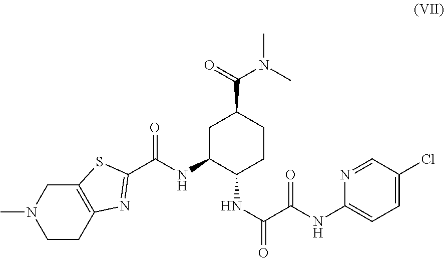
or a salt thereof, or a solvate thereof.

7. The stereoisomer according to claim 1, wherein the stereoisomer is:
N¹-(5-chloropyridin-2-yl)-N²-((1S,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VIII): 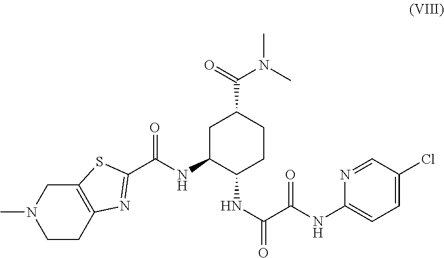
or a salt thereof, or a solvate thereof.

8. A method for inspecting impurities in a pharmaceutical composition containing N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (II): 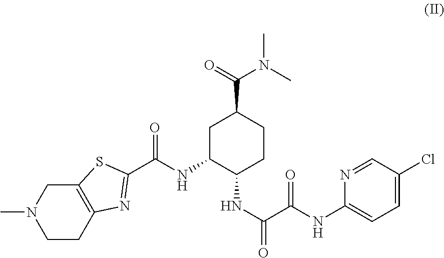
or a salt thereof, or a solvate thereof, comprising
using, as a standard, at least one or more compound(s), salt(s), or solvate(s) selected from the group consisting of:
(a) N¹-(5-chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (III): 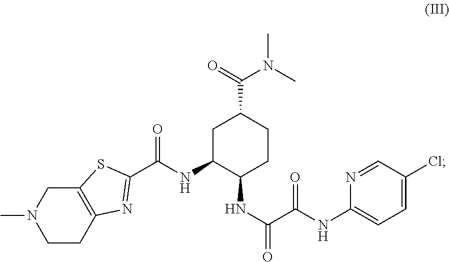
or a salt thereof, or a solvate thereof,
(b) N¹-(5-chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IV): 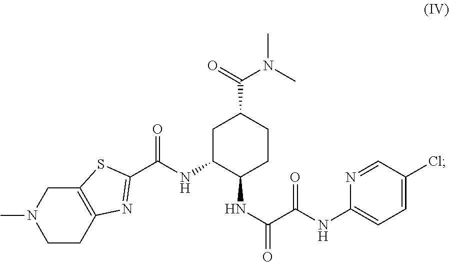
or a salt thereof, or a solvate thereof,
(c) N¹-(5-chloropyridin-2-yl)-N²-((1R,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (V): 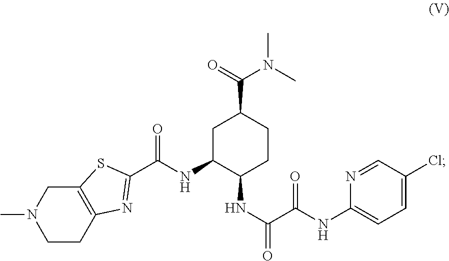
or a salt thereof, or a solvate thereof,
(d) N¹-(5-chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VI): 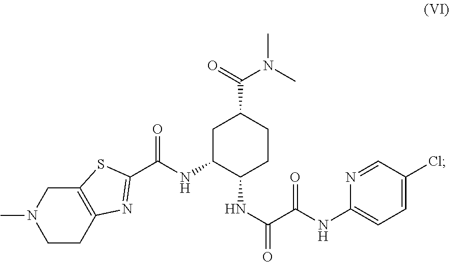
or a salt thereof, or a solvate thereof,
(e) N¹-(5-chloropyridin-2-yl)-N²-((1S,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VII): 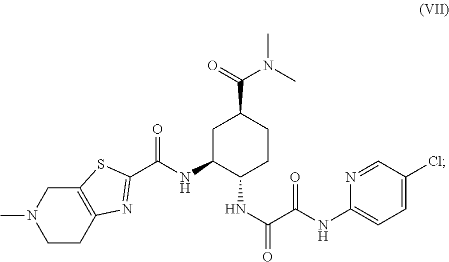
or a salt thereof, or a solvate thereof,
(f) N¹-(5-chloropyridin-2-yl)-N²-((1S,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VIII): 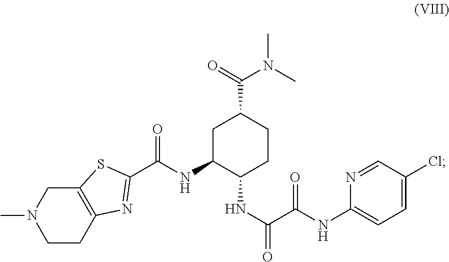
or a salt thereof, or a solvate thereof,
(g) N¹-(5-chloropyridin-2-yl)-N²-((1R,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IX): 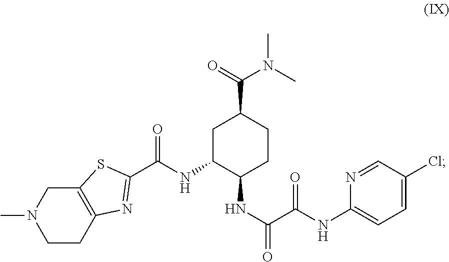
or a salt thereof, or a solvate thereof.

9. The method according to claim 8, wherein the standard is:
N¹-(5-chloropyridin-2-yl)-N²-((1R,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (III): 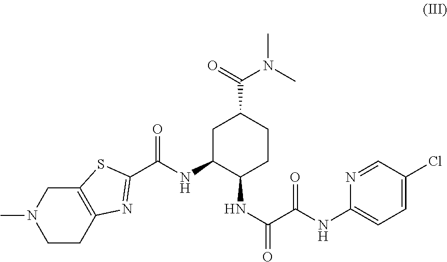
or a salt thereof, or a solvate thereof.

10. The method according to claim 8, wherein the standard is:
N¹-(5-chloropyridin-2-yl)-N²-((1R,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (IV): 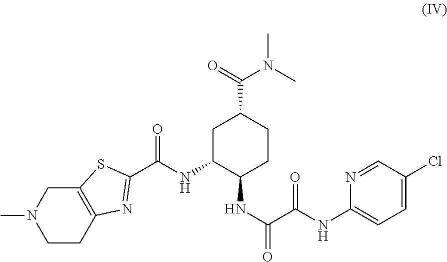
or a salt thereof, or a solvate thereof.

11. The method according to claim 8, wherein the standard is:
N¹-(5-chloropyridin-2-yl)-N²-((1R,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (V): 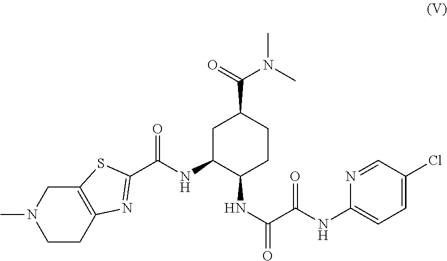
or a salt thereof, or a solvate thereof.

12. The method according to claim 8, wherein the standard is:
N¹-(5-chloropyridin-2-yl)-N²-((1S,2R,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VI): 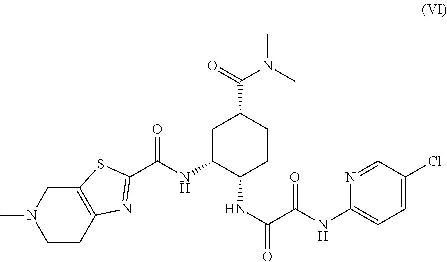
or a salt thereof, or a solvate thereof.

13. The method according to claim 8, wherein the standard is:
N¹-(5-chloropyridin-2-yl)-N²-((1S,2S,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VII): 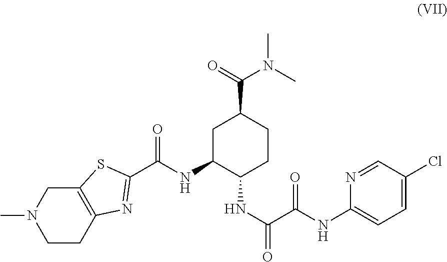
or a salt thereof, or a solvate thereof.

14. The method according to claim 8, wherein the standard is:
N¹-(5-chloropyridin-2-yl)-N²-((1S,2S,4R)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (VIII): 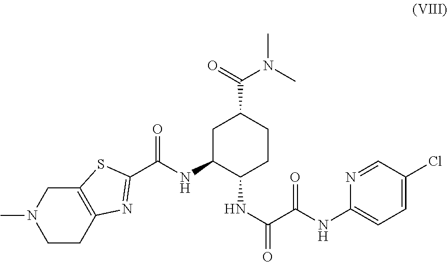
or a salt thereof, or a solvate thereof.

15. A substantially pure N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (II): 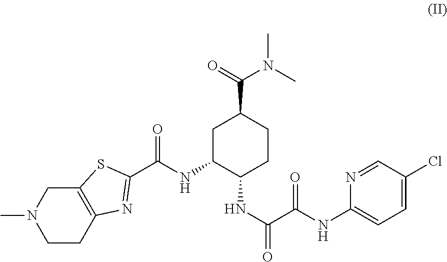
or a salt thereof, or a solvate thereof, wherein the compound II or salt, or solvate thereof is substantially free from a stereoisomer of compound II or salt thereof, or solvate thereof.

16. The compound or a salt thereof, or a solvate thereof according to claim 15, wherein the content of the stereoisomer or salt thereof, or solvate thereof is 3% or less.

17. A substantially pure pharmaceutical composition containing N¹-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide represented by the following formula (II): 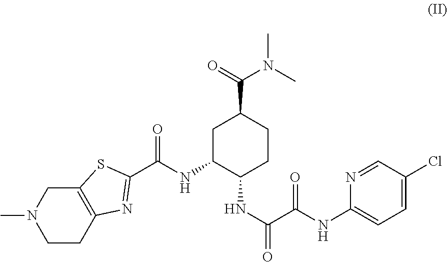
or a salt thereof, or a solvate thereof, wherein the composition is substantially free from stereoisomer of compound II or salt thereof, or solvate thereof.

18. The pharmaceutical composition according to claim 17, wherein the content of the stereoisomer or salt thereof, or solvate thereof is 3% or less.
